(19) United States
(12) Patent Application Publication
(10) Pub. No.: US 2019/0142566 A1
(43) Pub. Date: May 16, 2019

(54) Aortic Arch Embolic Protection Device

(71) Applicant: Yale University, New Haven, CT (US)

(72) Inventors: Alexandra Lansky, Rye, NY (US); Cody Pietras, New York, NY (US); Tarek Fahmy, New Haven, CT (US); Brendan Bell, Saratoga Springs, NY (US)

(73) Assignee: Yale University, New Haven, CT (US)

(21) Appl. No.: 16/099,624

(22) PCT Filed: May 10, 2017
(86) PCT No.: PCT/US17/32045

§ 371 Date: Nov. 7, 2018

Related U.S. Application Data
(60) Provisional application No. 62/334,085, filed on May 10, 2016.

(51) Int. Cl. A61F 2/01

(52) U.S. Cl. CPC A61F 2/01; A61F 2002/011; A61F 2230/0069; A61F 2230/0006; A61F 2002/018

(57) ABSTRACT

Advantageous instruments, assemblies and methods are provided for undertaking surgical procedures (e.g., cardiovascular interventional procedures). The present disclosure provides advantageous devices, assemblies and methods in the field of cardiac surgery and interventional cardiology procedures. Advantageous protection assemblies/devices (e.g., aortic arch embolic protection assemblies/devices) and related methods of use are provided. The present disclosure relates generally to embolic protection devices for use in the aortic arch to protect the distal vasculature during cardiac surgery and interventional cardiology procedures. An exemplary embolic protection device can take the form of an expandable/inflatable bladder member in a curved tube shape. The present disclosure provides advantageous (embolic) protection devices/assemblies, systems incorporating such devices/assemblies, and methods of use of such devices/assemblies for the benefit of such surgical practitioners and their patients.

### FIELD OF THE DISCLOSURE

The present disclosure relates generally to surgical equipment and procedures (e.g., in the field of cardiac surgery and interventional cardiology procedures) and, more particularly, to protection assemblies/devices (e.g., aortic arch embolic protection assemblies/devices) and related methods of use.

### BACKGROUND OF THE DISCLOSURE

Stroke is a devastating complication of cardiac surgical or endovascular procedures that occurs in up to 5-10% of patients and silent ischemic embolization has been reported to occur in about 70% to 100% of patients depending on the procedure. The presence of these clinically silent brain infarcts adds to ischemic brain burden and has been linked to dementia, cognitive decline, and an increased risk of subsequent overt stroke. It is estimated that up to 600,000 patients undergo cardiac surgical or endovascular procedures annually and are at risk for cerebral ischemia.

For example, stroke is a dreaded complication of endovascular procedures due to its association with an extreme morbidity and mortality burden. Periprocedural stroke rates increase with the complexity of cardiac surgery, ranging from about 1-5% for coronary artery bypass graft surgery (CABG) or isolated aortic valve replacement to as high as 7.4% for combined CABG and valve surgery and 9.7% for multiple valve surgery. Periprocedural stroke during catheter-based cardiovascular procedures is also a major concern. While stroke after left heart catheterization or percutaneous coronary intervention (PCI) is rare (less than 0.5%), it is associated with significant morbidity and an in-hospital mortality rate of about 25% to 30%.

Cerebral microembolism is the primary mechanism of periprocedural stroke during catheter-based interventions, and is primarily caused by embolization of aortic plaque dislodged during retrograde instrumentation of the aortic arch. In particular, retrograde catheterization with crossing of the aortic valve has been associated with focal diffusion-imaging abnormalities suggesting cerebral embolic events in about 22% of patients, in addition to a 3% rate of clinical neurological deficits.

In patients undergoing transcatheter aortic valve implantation (TAVI), about 3-4% of patients experience a stroke within 30 days, and 2-3% suffer a disabling stroke. Half of TAVI-related strokes are directly procedure-related and patients who experience a stroke after TAVI have more than a 3.5-fold increase in 30-day mortality. As during other types of cardiac procedures, periprocedural stroke during TAVI is generally ischemic and embolic. TAVI patients have several high-risk features that make cerebral embolization particularly common. First, the prevalence of severe aortic atherosclerosis increases across grades of AS, which when combined with the large-caliber catheters necessary for TAVI, make dislodgement of aortic debris more likely. Second, disruption of aortic valvular and annular calcification during TAVI is an additional source of embolic material; procedural transcranial Doppler monitoring indicates that the valve itself is the primary source of cerebral emboli following TAVI, and that most emboli are composed of debris dislodged during direct manipulation of the calcified aortic valve and crushing of the leaflets and aortic annulus during implantation.

In addition to periprocedural stroke, there is also evidence that clinically silent cerebral infarction, as detected on diffusion-weighted magnetic resonance imaging (DW-MRI), is common after a wide variety of cardiac interventions and surgical procedures. New foci of restricted cerebral perfusion on DW-MRI (cerebral ischemic lesions) are present in 58% to 93% after TAV. In mixed procedure populations (coronary artery bypass graft surgery [CABG], valve surgery, or combined procedures), new ischemic lesions on DW-MRI are detected in 15% to 63% of patients. This includes up to half of all patients after any CABG and approximately one third of patients after CABG without cardiopulmonary bypass. While the clinical significance of asymptomatic DW-MRI lesions after cardiac surgery is incompletely characterized, the presence of clinically silent brain infarcts has been associated with frailty, declines in physical function, reduced cognitive ability, depressive symptoms, and an increased risk of subsequent stroke or TIA in population-based studies.

Given the frequency and dire implications of periprocedural stroke and other embolic phenomena, methods to reduce cerebral embolism during cardiac interventions are sorely needed.

As shown in Table 1A below, a number of devices have been developed to attempt to mitigate the neuroembolic consequences observed during and after cardiac interventions and surgery. However, to date none have achieved widespread adoption.

TABLE 1A
Existing Embolic Deflection Devices:

| | Endovascular Devices | | | | |
|---|---|---|---|---|---|
| | | | Claret CE | | |
| | Keystone Heart | | Pro/Montage/ | Surgical Devices | |
| | TriGuard | Embrella | Sentinel Filter | Embol-X | EmBlocker |
| Regulatory | Investigational | CE-marked | CE-marked | CE-marked | CE-marked |
| status | | | | FDA approved | |
| Access | Transfemoral | Radial/brachial | Radial/brachial | Surgical | Surgical |
| | 9F (7F delivery) | 6F | 6F | (trans-aortic) | (thoracotomy) |
| Intended Use | TAVR | TAVR | TAVR | Cardiac surgery | Cardiac surgery |
| | | | | with CPB | |
| Vessel | Brachiocephalic & | Brachiocephalic | Brachiocephalic & | Ascending aortic | Brachiocephalic |
| Coverage | LCC & LSC | & LCC | LCC | lumen | & LCC |
| Protection | Deflection (filter) | Deflection | Capture | Capture | Deflection |
| Mechanism | | (filter) | (dual filter) | (filter) | (ultrasonic) |
| Filter | nitinol | polyurethane | polyurethane | polyester | NA |
| Material | | | | | |
| Pore Size | 130 × 250 μm | 100 μm | 140 μm | 120 μm | NA |

CPB = cardiopulmonary bypass;
LCC = left common carotid artery;
LSC = left subclavian artery;
NA = not applicable;
TAVR = transcatheter aortic valve replacement

There are several reasons for this lack of influence on conventional practice: existing devices are difficult to use, adding complexity to an already complex index procedure; most existing devices do not provide coverage of all cerebral vessels; problems with apposition to the aortic wall and difficulty in achieving device positioning, due to limited filter coverage and rigid frames; disturbance of aortic plaque and/or damage to the takeoff vessels (e.g., with devices that are introduced from above the aorta or require extension into the cerebral vessel for stabilization); and existing devices for use during percutaneous procedures generally require the use of a dedicated sheath, exposing the patient to additional risk for vascular complications.

The global embolic protection devices market was forecasted to reach US $0.54 billion by 2015, with 55% of this accounted for catheter occlusion and filter devices (Global Embolic Protection Devices [EPD]—Market Growth Analysis, 2009-2015), and the remainder by balloon occlusion devices. The majority of current EPD use was in the coronary and carotid interventions; however, the increasing adoption of minimally-invasive approaches to valvular and structural heart disease, including TAVI, as well as their associated rates of neurological injury, indicated increasing demand for embolic protection during these procedures was likely.

Previous devices for attempting to prevent cerebral embolism by means of a filter placed in the aortic arch are described and disclosed in U.S. Pat. No. 8,728,114, and U.S. Patent Pubs. Nos. 2012/0109182; 2014/0074148; 2003/0100940; 2009/0254172; 2004/0024416 and 2010/0179647, the entire contents of each being hereby incorporated by reference in their entireties.

As such, a need exists among end-users and/or manufacturers to develop protection assemblies/devices (e.g., embolic protection assemblies/devices) that include improved features/structures. In addition, a need remains for instruments, assemblies and methods that allow embolic protection through designs and techniques that are easily understood and implemented by surgical personnel.

Thus, an interest exists for improved protection assemblies/devices (e.g., embolic protection assemblies/devices) and related methods of use. These and other inefficiencies and opportunities for improvement are addressed and/or overcome by the devices, assemblies and methods of the present disclosure.

### SUMMARY OF THE DISCLOSURE

According to the present disclosure, advantageous instruments, assemblies and methods are provided for undertaking surgical procedures (e.g., cardiovascular interventional procedures). The present disclosure provides advantageous devices, assemblies and methods in the field of cardiac surgery and interventional cardiology procedures. More particularly, the present disclosure provides advantageous protection assemblies/devices (e.g., aortic arch embolic protection assemblies/devices) and related methods of use.

In general, the present disclosure relates generally to embolic protection devices for use in the aortic arch to protect the distal vasculature during cardiac surgery and interventional cardiology procedures. In some embodiments, disclosed herein is an exemplary expandable/collapsible neuro-protection device utilized during cardiological intervention (e.g., an endovascular retrievable protecting device for neuro-protection during vascular or aortic arch surgery).

The neuro-protection device can be utilized as an endovascular retrievable filter positioned in the aortic arch, across the major cerebral vessels, to protect the brain from embolization of embolic debris and stroke during endovascular or surgical cardiac or ascending aortic arch procedures or for chronic use in patients at high risk for cerebral embolization and stroke. In certain embodiments, the present disclosure provides a tubular protecting device that inserts in the aortic arch and is expandable during the application and collapsible for removal.

In some embodiments, the present disclosure provides for a protection device taking the form of a polymer mesh filter configured into an elongated tubular pattern, the mesh filter including a proximal portion, a distal portion, and being configured/creased in a pattern such that when forced apart from the proximal to distal end, the mesh filter is in an expanded state and when compressed it is in a collapsed state. Deployment is for example by mechanical force on one or more ends (proximal or distal), thereby allowing the tube to expand in place. Removal can be via a push mechanism that collapses the tube. Collapse occurs along the crease lines of the deployable mesh filter.

One exemplary embolic protection device is an expandable/collapsible polymer mesh filter in a curved tube shape. The mesh is configured/creased in a pattern such that when opposing outward forces are applied to the proximal and distal ends along the cylinder/tube axis, the filter self-expands to adjust to the contours of the individual patient's aortic arch, allowing blood to flow normally but preventing clinically significant emboli from entering the cerebral takeoff vessels in the aortic arch. When reversing forces are applied, the device/filter collapses into its initial compressed state for low-profile introduction to and/or retrieval from the body.

In certain embodiments, the present disclosure provides for an origami-based expandable/collapsible filter device (e.g., an expandable/collapsible coronary filter based on the principles of origami).

In certain embodiments, the present disclosure provides for a single-use, biocompatible filter device fabricated from polymeric material (e.g., polyethylene, polypropylene, biodegradable polymers, polyesters, etc.). The filter device can be delivered through a transfemoral arterial access (e.g., via 7 French catheter or less), positioned across the aortic arch, and anchored in position by deployment members (e.g., wires attached to nitinol rings). The filter portion of the device covers all three major cerebral arteries in the aortic arch (innominate, left common carotid and subclavian), maintaining blood flow to the cerebral vessels through pores (e.g., 100 μm pores) while deflecting larger emboli to the descending aorta. The device is intended to reduce the passage of embolic material (debris/thrombus) to the cerebral arteries during endovascular or surgical cardiac or aortic procedures or in patients at high risk for cerebral embolization.

In other embodiments, the present disclosure provides for an inflatable/expandable and deflatable/collapsible protection device (e.g., neuro-protection device) utilized during surgical procedures (e.g., during cardiological intervention).

Thus, the present disclosure provides, inter alia, advantageous protection devices/assemblies, systems incorporating such devices/assemblies, and methods of use of such devices/assemblies for the benefit of such surgical practitioners and their patients.

The present disclosure provides for a protection assembly including a bladder member extending from a proximal end to a distal end, the bladder member having an outer wall and an inner wall sealed together to form an inflatable cavity between the outer and inner walls, the bladder member defining a window portion; a porous filter material mounted to the bladder member, the porous filter material extending across and covering the window portion; wherein after the bladder member is positioned in a desired anatomical location, introduction of fluid to the bladder member causes the bladder member to expand from a collapsed position to an expanded position.

The present disclosure also provides for a protection assembly wherein after the bladder member is positioned in the expanded position, removal of fluid from the bladder member causes the bladder member to collapse from the expanded position to the collapsed position.

The present disclosure also provides for a protection assembly wherein when the bladder member is expanded from the collapsed position to the expanded position, the outer wall of the expanded bladder member is configured and dimensioned to substantially conform to an aortic wall of the desired anatomical location. The present disclosure also provides for a protection assembly wherein when the bladder member is moved from the collapsed position to the expanded position, the porous filter material is configured and dimensioned to substantially cover three major cerebral arteries in an aortic arch of the desired anatomical location.

The present disclosure also provides for a protection assembly wherein the porous filter material includes a plurality of pores, each pore of the plurality of pores having a pore size of from about 100 μm to about 150 μm.

The present disclosure also provides for a protection assembly wherein the bladder member is hollow and substantially tubular. The present disclosure also provides for a protection assembly wherein a top side of the bladder member defines the window portion, the window portion rectangular in shape.

The present disclosure also provides for a protection assembly further including a top tether member and a bottom tether member attached to the distal end of the bladder member, the top tether member including a fill hose through which fluid is moved in order to inflate or deflate the bladder member; and wherein the bottom tether member is attached to a support member, the support member extending from the distal end of the bladder member to the proximal end of the bladder member. The present disclosure also provides for a protection assembly wherein the support member is a semi-rigid rod or wire that is attached to the bladder member at the distal end and the proximal end of the bladder member. The present disclosure also provides for a protection assembly wherein the inner and outer walls of the bladder member include a plurality of sealed perforations therethrough.

The present disclosure also provides for a method for performing a procedure, including providing a bladder member extending from a proximal end to a distal end, the bladder member having an outer wall and an inner wall sealed together to form an inflatable cavity between the outer and inner walls, the bladder member defining a window portion, with a porous filter material mounted to the bladder member, the porous filter material extending across and covering the window portion; positioning the bladder member in a desired anatomical location; and introducing fluid to the bladder member to cause the bladder member to expand from a collapsed position to an expanded position.

The present disclosure also provides for a method for performing a procedure wherein after the bladder member is positioned in the expanded position, removal of fluid from the bladder member causes the bladder member to collapse from the expanded position to the collapsed position.

The present disclosure also provides for a method for performing a procedure wherein when the bladder member is expanded from the collapsed position to the expanded position, the outer wall of the expanded bladder member is configured and dimensioned to substantially conform to an aortic wall of the desired anatomical location. The present disclosure also provides for a method for performing a procedure wherein when the bladder member is moved from the collapsed position to the expanded position, the porous filter material is configured and dimensioned to substantially cover three major cerebral arteries in an aortic arch of the desired anatomical location.

The present disclosure also provides for a method for performing a procedure wherein the porous filter material includes a plurality of pores, each pore of the plurality of pores having a pore size of from about 100 μm to about 150 μm.

The present disclosure also provides for a method for performing a procedure wherein the bladder member is hollow and substantially tubular. The present disclosure also provides for a method for performing a procedure wherein a top side of the bladder member defines the window portion, the window portion rectangular in shape.

The present disclosure also provides for a method for performing a procedure further including a top tether member and a bottom tether member attached to the distal end of the bladder member, the top tether member including a fill hose through which fluid is moved in order to inflate or deflate the bladder member; and wherein the bottom tether member is attached to a support member, the support member extending from the distal end of the bladder member to the proximal end of the bladder member. The present disclosure also provides for a method for performing a procedure wherein the support member is a semi-rigid rod or wire that is attached to the bladder member at the distal end and the proximal end of the bladder member. The present disclosure also provides for a method for performing a procedure wherein the inner and outer walls of the bladder member include a plurality of sealed perforations therethrough.

The present disclosure also provides for a protection assembly including a hollow and tubular bladder member extending from a proximal end to a distal end, the bladder member having an outer wall and an inner wall sealed together to form an inflatable cavity between the outer and inner walls, a top side of the bladder member defining a rectangular window portion; a porous filter material mounted to the bladder member, the porous filter material extending across and covering the rectangular window portion, the porous filter material including a plurality of pores, each pore of the plurality of pores having a pore size of from about 100 μm to about 150 μm; a top tether member and a bottom tether member attached to the distal end of the bladder member, the top tether member including a fill hose through which fluid is moved in order to inflate or deflate the bladder member, and the bottom tether member attached to a support member, the support member extending from the distal end of the bladder member to the proximal end of the bladder member; wherein after the bladder member is positioned in a desired anatomical location, introduction of fluid to the bladder member causes the bladder member to expand from a collapsed position to an expanded position; wherein after the bladder member is positioned in the expanded position, removal of fluid from the bladder member causes the bladder member to collapse from the expanded position to the collapsed position; wherein when the bladder member is expanded from the collapsed position to the expanded position, the outer wall of the expanded bladder member is configured and dimensioned to substantially conform to an aortic wall of the desired anatomical location; and wherein when the bladder member is moved from the collapsed position to the expanded position, the porous filter material is configured and dimensioned to substantially cover three major cerebral arteries in an aortic arch of the desired anatomical location.

Any combination or permutation of embodiments is envisioned. Additional advantageous features, functions and applications of the disclosed systems, assemblies and methods of the present disclosure will be apparent from the description which follows, particularly when read in conjunction with the appended figures. All references listed in this disclosure are hereby incorporated by reference in their entireties.

### BRIEF DESCRIPTION OF THE DRAWINGS

Features and aspects of embodiments are described below with reference to the accompanying drawings, in which elements are not necessarily depicted to scale.

Exemplary embodiments of the present disclosure are further described with reference to the appended figures. It is to be noted that the various features, steps and combinations of features/steps described below and illustrated in the figures can be arranged and organized differently to result in embodiments which are still within the scope of the present disclosure. To assist those of ordinary skill in the art in making and using the disclosed systems, assemblies and methods, reference is made to the appended figures, wherein:

FIGS. 1A-1B are side views of an exemplary protection device according to the present disclosure—FIG. 1A shows the device in an initial collapsed state, and FIG. 1B shows the device in an expanded state;

FIG. 2 is a side view of the device of FIG. 1A, with two deployment wires releasably attached to the device;

FIGS. 3A-3C are side views of the device of FIG. 1A-1B—FIG. 3A shows the device in an initial collapsed state, FIG. 3B shows the device in an expanded state, and FIG. 3C shows the device returned to the collapsed state;

FIG. 3D is side view of the two deployment wires of FIGS. 2 and 3A-3B;

FIG. 4A is side view of the device of FIG. 1A, prior to deployment;

FIG. 4B is side view of the device of FIG. 4A, after deployment;

FIG. 4C is side view of the device of FIG. 4B, prior to retraction;

FIG. 5 is a side view of another exemplary protection assembly according to the present disclosure, with the protection device in an initial collapsed state/configuration;

FIG. 6 is a side view of the protection device of FIG. 5 in an inflated and expanded state/configuration;

FIGS. 7-8 are side views of another exemplary protection assembly according to the present disclosure, with the protection device in an inflated and expanded state;

FIG. 9 is a side view of another exemplary protection assembly according to the present disclosure, with the protection device in an inflated and expanded state;

FIGS. 10A-10D depict various aortic arch anatomic types;

FIGS. 11A-11C depict various cerebral vessel takeoff positioning types;

FIG. 12 is a side view of another exemplary protection assembly according to the present disclosure;

FIG. 13 is a side view of the protection device of FIG. 12 in an inflated and expanded state/configuration;

FIGS. 14-15 are side perspective views of another exemplary protection assembly according to the present disclosure; and

FIG. 16 is a side view of the protection device of FIG. 14.

### DETAILED DESCRIPTION OF DISCLOSURE

The exemplary embodiments disclosed herein are illustrative of advantageous protection assemblies/devices (e.g., embolic protection assemblies/devices), and systems of the present disclosure and methods/techniques thereof. It should be understood, however, that the disclosed embodiments are merely exemplary of the present disclosure, which may be embodied in various forms. Therefore, details disclosed herein with reference to exemplary protection devices/fabrication methods and associated processes/techniques of assembly and use are not to be interpreted as limiting, but merely as the basis for teaching one skilled in the art how to make and use the advantageous protection devices/systems and/or alternative protection devices/assemblies of the present disclosure.

The present disclosure provides improved systems, assemblies and methods in the field of cardiac surgery and interventional cardiology procedures. In general, the present disclosure provides advantageous protection assemblies/devices (e.g., aortic arch embolic protection assemblies/devices) and related methods of use. The present disclosure provides, inter alia, advantageous protection devices/assemblies (e.g., embolic protection devices/assemblies), systems incorporating such devices/assemblies, and methods of use of such devices/assemblies for the benefit of such surgical practitioners and their patients.

In exemplary embodiments, the present disclosure relates generally to embolic protection devices for use in the aortic arch to protect the distal vasculature during cardiac surgery and interventional cardiology procedures. For example, the present disclosure provides for an expandable/collapsible neuro-protection device utilized during cardiological intervention (e.g., an endovascular retrievable protecting device for neuro-protection during vascular or aortic arch surgery).

In other embodiments, the present disclosure provides for an inflatable/expandable and deflatable/collapsible neuro-protection device utilized during cardiological intervention.

In certain embodiments, the embolic protection device is an expandable/collapsible mesh filter (e.g., polymer mesh filter) in a curved tube shape. For example, the mesh can be configured/creased in a pattern such that when opposing outward forces are applied to the proximal and distal ends along the cylinder/tube axis, the filter self-expands to adjust to the contours of the individual patient's aortic arch, allowing blood to flow normally but preventing clinically significant emboli from entering the cerebral takeoff vessels in the aortic arch. Moreover, when reversing forces are applied, the device/filter collapses into its initial compressed state for low-profile introduction to and/or retrieval from the body.

In some embodiments, the present disclosure provides for an origami-based expandable/collapsible filter device (e.g., an expandable/collapsible coronary filter based on the principles of origami).

Referring now to the drawings, like parts are marked throughout the specification and drawings with the same reference numerals, respectively. Drawing figures are not necessarily to scale and in certain views, parts may have been exaggerated for purposes of clarity.

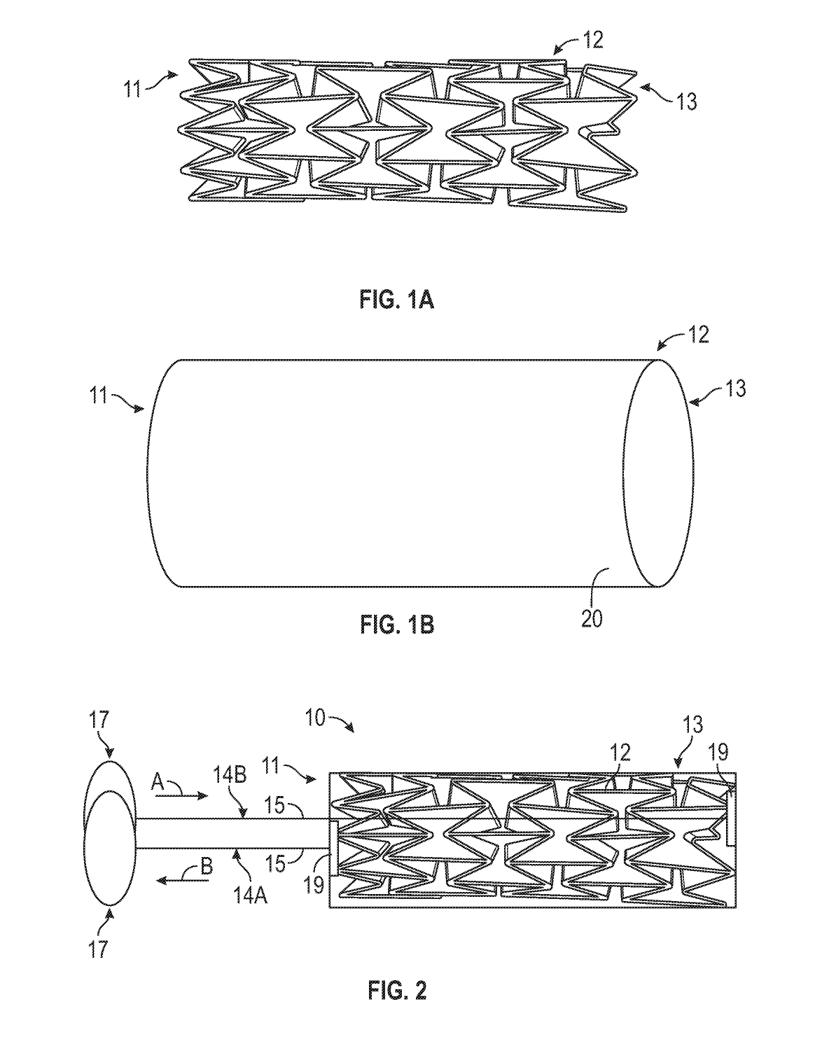

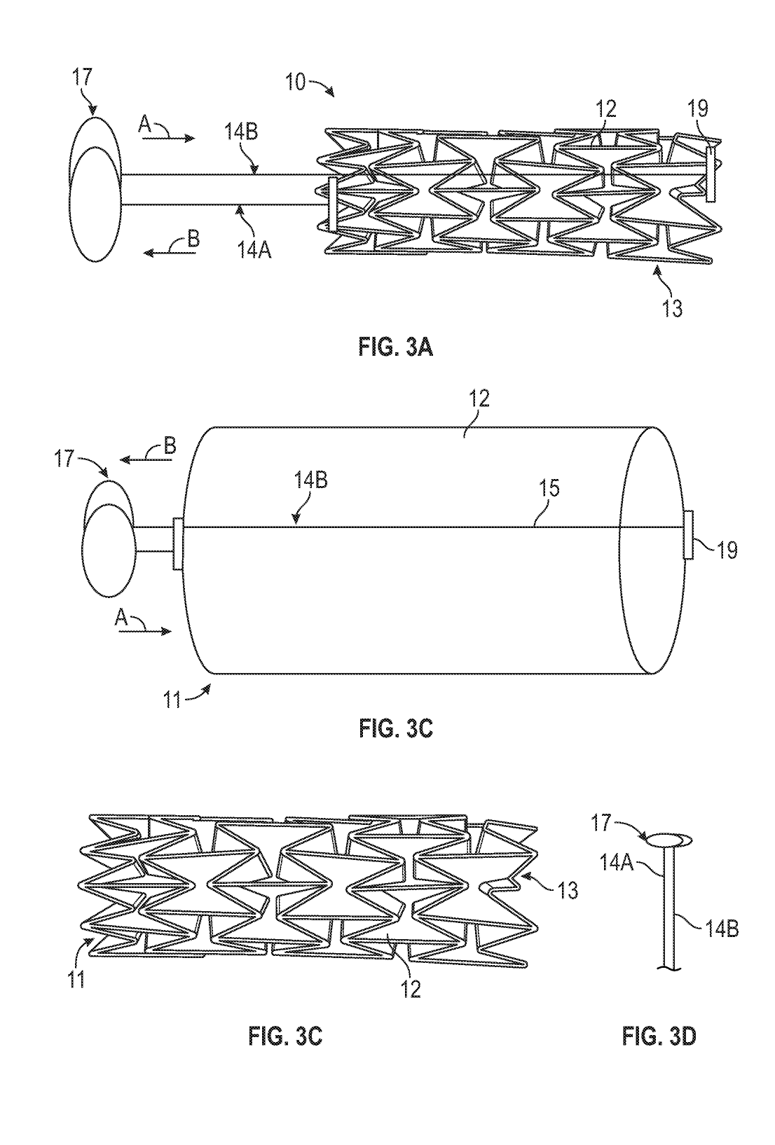

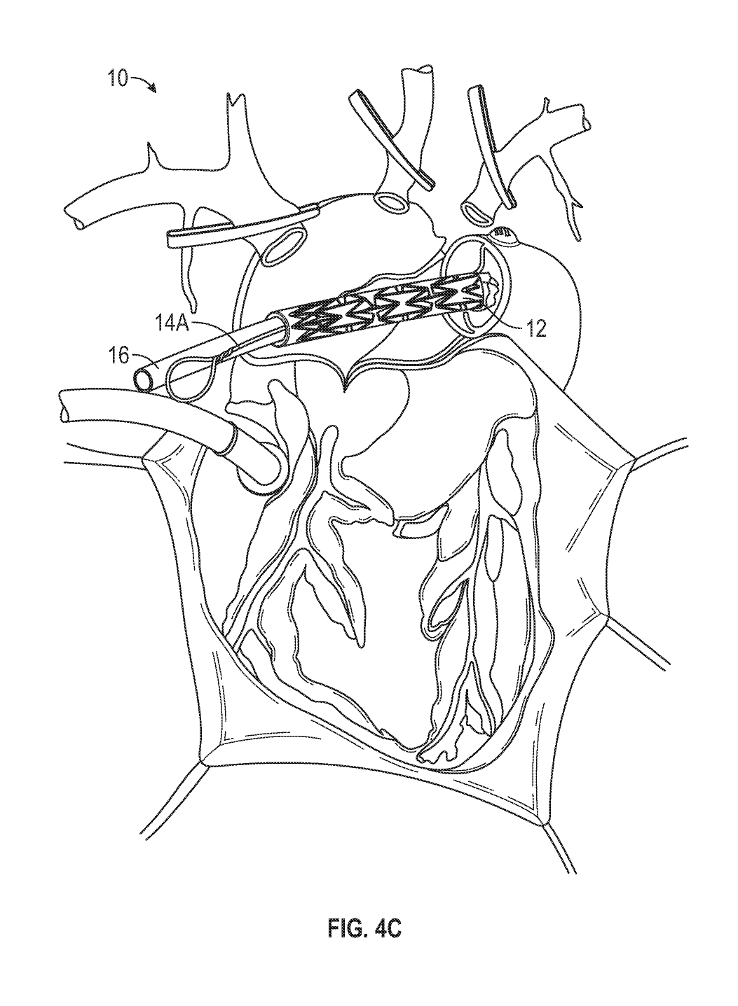

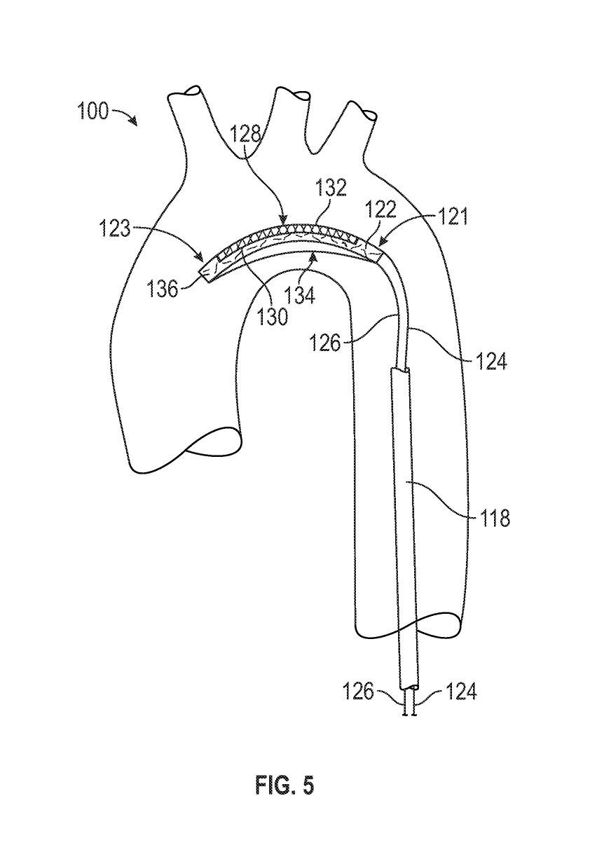

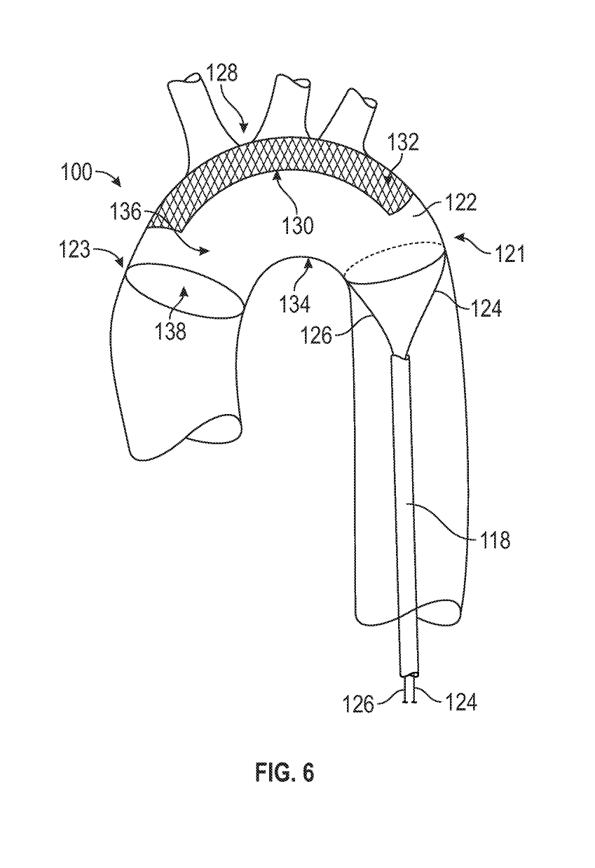

Referring now to FIGS. 1A-4C, an exemplary protection assembly 10 (e.g., embolic protection assembly 10) generally includes a protection device 12 (e.g., embolic protection device 12), one or more deployment members 14A, 14B, a delivery member 16 and an access or introducer sheath 18 (FIG. 4A). In general, access/introducer sheath 18 is typically configured and dimensioned to allow catheters (e.g., delivery catheter 16), stents, and other interventional/surgical devices (e.g., protection device 12) to be introduced into blood vessels.

For example and as shown in FIG. 4A, the sheath 18 (e.g., with an associated guidewire) may be introduced to a desired anatomical location/region. Thereafter, additional instrumentation and/or devices (e.g., delivery catheter 16, device 12) may be introduced to the anatomical location/region using a guidewire as a guide.

Exemplary deployment members 14A, 14B include a wire section 15, a handle section 17 (e.g, ring-shaped handle 17), and a fastener member 19 (e.g., pin-like fasteners 19). In some embodiments, at least portions of deployment members 14A, 14B are fabricated from nitinol or the like, although the present disclosure is not limited thereto. Rather, members 14A, 14B can be fabricated from a variety of materials or combination of materials.

In exemplary embodiments, the protection device 12 of assembly 10 takes the form of an expandable/collapsible polymer mesh-filter tubular device 12. As discussed further below, releasably secured deployment members 14A, 14B attached to the proximal and distal ends 11, 13 of the hollow tubular protection device 12 allow a user to control expansion and collapse of device 12. As shown in FIG. 4A, exemplary delivery device 16 takes the form of a catheter-based delivery device 16 configured and dimensioned for introducing the protection device 12 into the body, guiding the device 12 to a desired deployment location (e.g., trans-arterially), and retrieving the device 12 when the procedure (e.g., index procedure) is complete.

For example, at the beginning of an index procedure, the protection device 12 and releasably secured deployment members 14A, 14B are introduced into the femoral artery via the delivery catheter 16 passing through an introducer sheath 18 (e.g., either the index procedure sheath 18, or a dedicated sheath 18 contralateral to the index procedure introduction site) and advanced to the aortic arch. Once in place, the delivery catheter 16 is withdrawn, exposing the collapsed device 12 in place.

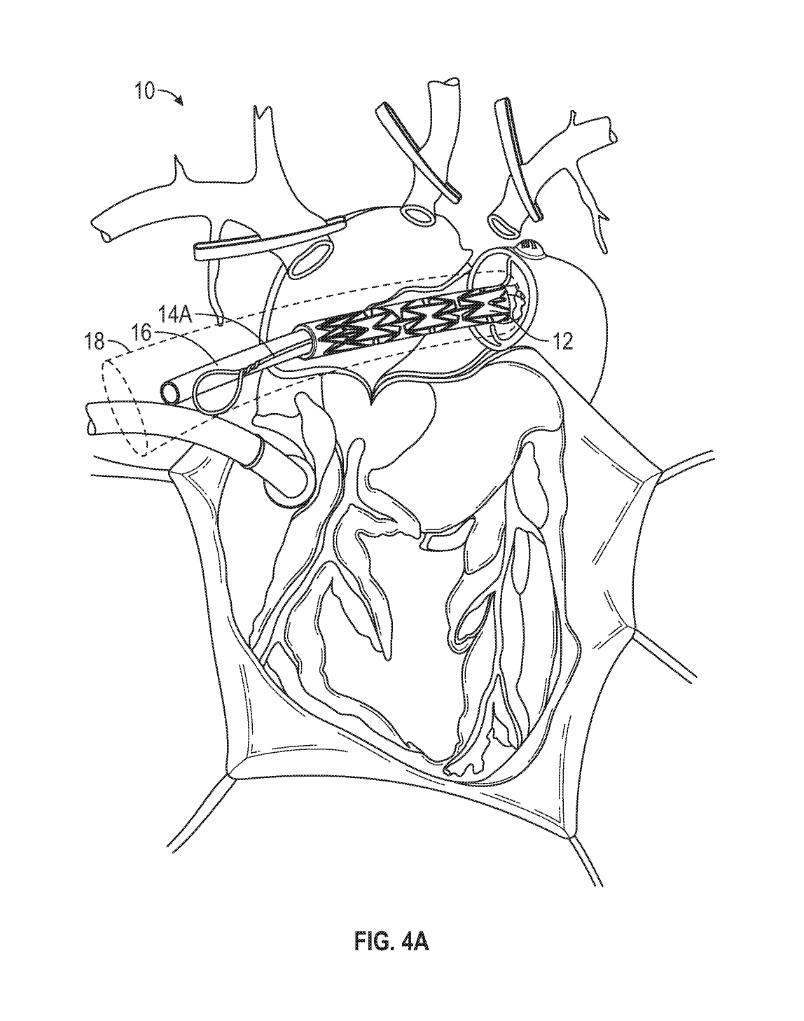

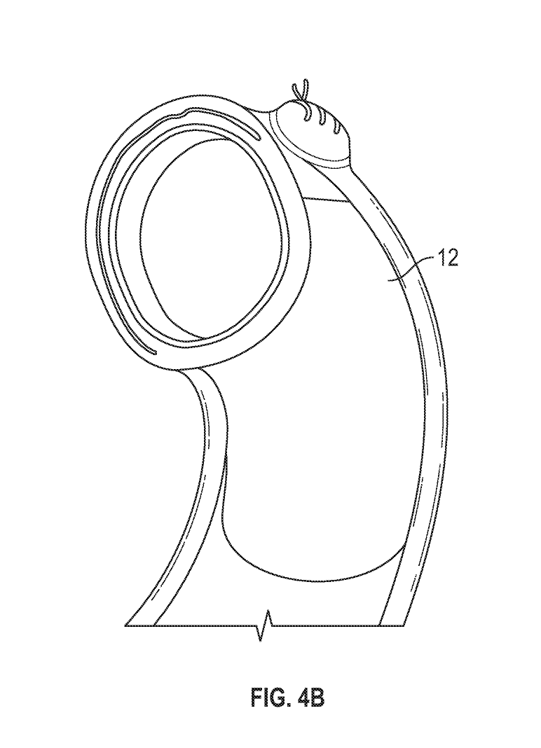

After the operator confirms (e.g., via fluoroscopy) that the device 12 is properly positioned, the device 12 can then be expanded by applying counter-traction or force to the two deployment members 14A, 14B. For example and as shown in FIG. 3A, the operator can push or move deployment member 14B via handle 17 in the direction of Arrow A, and can pull or move the deployment member 14A via handle 17 in the direction of Arrow B to deploy or expand the device 12 (as shown in FIG. 3B).

The mesh-filter tubular device 12 will expand to oppose the walls of the aortic arch (FIG. 4B), allowing blood to flow normally to the cerebral vessels through the aortic arch takeoffs, while preventing embolic material larger than the pore size of the expanded filter device 12 from passing to the brain. If the filter device 12, upon expansion, is not positioned properly to provide protection to the cerebral vessels, the device 12 may be collapsed, re-positioned, and re-expanded.

It is noted that deployment members 14A, 14B are depicted. However, deployment of device 12 can be obtained via other mechanical force on one or more ends 11, 13, thereby allowing the device 12 to expand in place. Removal/collapse of device 12 can be via a push mechanism that collapses the device 12. In general, collapse occurs along the crease lines of the deployable mesh-filter device 12.

Referring to FIG. 4B, the device 12 can remain in place for the duration of the index procedure. When the index procedure is complete and as depicted in FIG. 3B, the deployment members 14A, 14B are used to collapse the protection device 12 by pulling deployment member 14B via handle member 17 in the direction of Arrow B (member 14B includes the fastener member 19 positioned at distal end 13, or proximal to the heart), and by pushing deployment member 14A via handle 17 in the direction of Arrow A (member 14A includes the fastener member 19 positioned at proximal end 11 or distal to the heart). The device 12 will then return to the collapsed position, as shown in FIG. 3C. As shown in FIG. 4C, the device 12 can then be retrieved into the delivery catheter 16 and removed from the body with the delivery catheter 16 and sheath 18.

Exemplary protection device 12 takes the form of a collapsible tube filter device 12 having a slight curvature that is easily deployed and retracted in the vessel wall or aortic arch.

As such, device 12 is designed to be deployed and expanded as cylindrical filter device 12 that fills the vessel wall, allowing for substantially complete blood flow with substantially no leakage around the periphery or sides of the tube device 12.

Another advantageous aspect is the design/steps by which the device 12 is retrieved by collapsing the tubular device 12 post-surgery. As noted, device 12 can be utilized as a neuro-protection device 12 implemented as an endovascular retrievable filter device 12 positioned in the aortic arch, across the major cerebral vessels, to protect the brain from embolization of embolic debris and stroke during endovascular or surgical cardiac or ascending aortic arch procedures, or for chronic use in patients at high risk for cerebral embolization and stroke.

As noted, device 12 can take the form of a deployable expanding/contracting cylindrical/tubular filter device that can be utilized as an endovascular filter device 12.

In exemplary embodiments and as shown in FIG. 1A, device 12 includes a triangulated cylindrical construction in which the filter material of device 12 is folded in a pattern enabling facile expansion of the filter device 12 to fit the particular vessel in which it is deployed. The same device 12 can be removed using reversed mechanical actuation in which the triangulated patterns essentially fold back and the tubular filter device 12 is contracted to the same size before surgical implantation (FIGS. 3A to 3C).

In certain embodiments, device 12 is a single-use, biocompatible filter device 12 fabricated from a polymeric filter mesh material. Device 12 is delivered through a transfemoral arterial access (e.g., via a 7 French catheter/sheath 18 or less), positioned across the aortic arch, and anchored/deployed in position by movement of deployment members 14A, 14B, as discussed above and as shown in FIG. 4B.

As also depicted in FIG. 4B, expanded filter device 12 is configured and dimensioned to cover all three major cerebral arteries in the aortic arch (innominate, left common carotid and subclavian), while maintaining blood flow to the cerebral vessels through its filter pores 20 (e.g., 100 μm pores) while deflecting larger emboli to the descending aorta. As such, device 12 can be advantageously utilized to reduce the passage of embolic material (debris/thrombus) to the cerebral arteries during endovascular or surgical cardiac or aortic procedures or in patients at high risk for cerebral embolization.

In certain embodiments, device 12 is fabricated from, at least in part, FDA approved polymer mesh material having surfaces that are triangulated/folded/creased in a pattern (e.g., based on triangulation of a cylindrical mesh of filter material—FIG. 1A) allowing for device 12 expansion and collapse.

For example, the polymer mesh material of device 12 can be configured into an elongated tubular pattern that includes a proximal portion 11, a distal portion 13, and that is configured/creased in a pattern such that when forced apart from the proximal 11 to distal end 13 it is in an expanded state (FIG. 1B) and when compressed it is in collapsed initial state (FIG. 1A). Device 12 can be fabricated by triangulating/folding rectangular mesh material into indented creases before seaming/sealing the sides to form a tube device 12 (FIG. 1A).

In certain embodiments, the device in the folded/collapsed state (FIG. 1A) is a small cylinder/tube (approximately 10 cm in length and 1 cm in diameter). Attachment of deployment members 14A, 14B (e.g., via releasable fastener members or pins 19) are configured to stress the folded/collapsed device 12 in opposite directions facilitating a facile method for expanding the device 12 during surgery, as discussed above. The same stress points can be reversed post-surgery, thereby collapsing the device 12 into the folded/collapsed state (e.g., for removal from the vessel wall). Because the device 12 expands gradually and gently when stress is applied to opposite ends via the members 14A, 14B, there is advantageously little risk associated with impingement or scarring of the vessel wall.

The mesh material of device 12 may fall in the class of degradable or non-degradable polymer matrices. The polymeric matrix material of the device 12 may be formed from one or more polymers or copolymers. By varying the composition and morphology of the polymeric matrix, one can achieve a variety of elasticities and tensile strengths permitting structural expansion and contraction.

The polymeric matrix material of device 12 may be formed from non-biodegradable or biodegradable polymers; however, preferably, the polymeric matrix material is non-biodegradable. The polymeric matrix material can be selected to degrade over a time period from ranging from one day to one year or longer.

In general, synthetic polymers are preferred, although natural polymers may be used. Representative polymer mesh that degrade and may be used as scaffolding materials for device 12 include poly(lactic acid), poly(glycolic acid), poly(lactic acid-co-glycolic acids), polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); poly(glycolide-co-caprolactones); polycarbonates such as tyrosine polycarbonates; polyamides (including synthetic and natural polyamides), polypeptides, and poly(amino acids); polyesteramides; other biocompatible polyesters; poly(dioxanones); poly(alkylene alkylates); hydrophilic polyethers; polyurethanes; polyetheresters; polyacetals; polycyanoacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), polyvinyl alcohols, polyvinylpyrrolidone; poly(alkylene oxides) such as polyethylene glycol (PEG); derivativized celluloses such as alkyl celluloses (e.g., methyl cellulose), hydroxyalkyl celluloses (e.g., hydroxypropyl cellulose), cellulose ethers, cellulose esters, nitrocelluloses, polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), as well as derivatives, copolymers, and blends thereof.

As used herein, “derivatives” include polymers having substitutions, additions of chemical groups and other modifications to the polymeric backbones described above routinely made by those skilled in the art. Natural polymers, including proteins such as albumin, collagen, gelatin, prolamines, such as zein, and polysaccharides such as alginate and pectin, may also be incorporated into the polymeric matrix material. While a variety of polymers may be used to form the polymeric matrix material, generally, the resulting polymeric matrix material will be an elastic material.

The polymeric matrix material may be formed from polymers having a variety of molecular weights. Generally, the polymers which make up the polymeric matrix material possess average molecular weights of about 500 Da or above. In some cases, the polymeric matrix material of device 12 is formed from an aliphatic polyester or a block copolymer containing one or more aliphatic polyester segments.

As noted, protection device 12 can be deployed in the aortic arch in a collapsed state. A simple mechanical maneuver involving mechanical actuation (e.g., via members 14A, 14B) deploys the device 12 (e.g., cylindrical tube filter device 12), and expands device 12 in the arch covering all three major cerebral arteries in the aortic arch (innominate, left common carotid and subclavian).

In exemplary embodiments, device 12 is an open tube device 12 that is scaffolded by a polymeric mesh material with a minimum of 100 um pores 20 (FIG. 1B). As such, device 12 maintains blood flow to the cerebral vessels through pores 20 (e.g., 100 μm pores) while deflecting larger emboli to the descending aorta. Exemplary device 12 is elastic and substantially fills the entire arch region preventing circumferential blood flow. On procedure completion another mechanical actuation (e.g., via members 14A, 14B) involving a reversal of steps during deployment contracts the filter mesh device 12. In certain embodiments, device 12 will entrap debris that is extracted and concentrated in the device 12 upon contraction and removal after surgery.

Compared to conventional protection devices (e.g., aortic arch embolic protection devices), the exemplary assemblies 10 and devices 12 of the present disclosure offer many advantages.

One advantageous function of assembly 10 and/or device 12 is that the self-expanding, self-adjusting nature of the sleeve-like device 12 in its expanded state allows the device 12 to substantially conform to the aortic wall, preventing the lack of apposition observed with metallic mesh or rigid frame devices. Lack of apposition can allow blood and embolic material to flow between other conventional filters and the upper wall of the aortic arch, leading to cerebral embolization or loss of device/filter positioning.

Another advantageous function of assembly 10 and/or device 12 is that the compliant material used to construct the device 12 reduces the risk of vascular injury during deployment, protection, or retrieval, compared with existing rigid frame devices and devices with protruding elements.

Another advantageous function of assembly 10 and/or device 12 is that the novel folding pattern of device 12 enables the device 12 to have a reduced delivery profile, reducing the risk of vascular injury during device deployment and retrieval.

Another advantageous function of assembly 10 and/or device 12 is the ease of delivery of device 12, which does not require precise positioning, allows faster deployment and less interference with the index procedure.

Another advantageous function of assembly 10 and/or device 12 is that the material composite of the device 12 enables the entire device 12 to be radio-opaque, enabling easy visualization of the entire device 12 on procedural fluoroscopy, without the need for specialized marker elements.

Another advantageous function of assembly 10 and/or device 12 is that the device 12 protects all cerebral vessel takeoffs in the aortic arch, unlike some other devices that do not provide complete protection.

Another advantageous function of assembly 10 and/or device 12 is that device 12 provides coverage for agnostic to aortic arch anatomic variants, and there is no reliance on interaction with takeoff vessels for positioning or stability.

Another advantageous function of assembly 10 and/or device 12 is that device 12 is delivered transfemorally, avoiding instrumentation of the supra-aortic arch vessels that could cause additional cerebral embolization.

Another advantageous function of assembly 10 and/or device 12 is that device 12 is held in position by radial outward force across the entire surface area of the device 12, reducing the pressure on any individual point and reducing the risk of endothelial injury, in comparison with conventional devices that utilize anchors or that focus pressure on the small surface area of a rigid frame.

Another advantageous function of assembly 10 and/or device 12 is that compared with conventional embolic capture devices, exemplary device 12 can be configured, in certain embodiments, to only deflect emboli, avoiding the risk that the filter/device 12 could become overwhelmed and reduce blood flow.

It is noted that there are no currently approved neuro-protection or deflection devices in the United States. Current neuro-protection devices under development are limited by providing incomplete cerebral vessel coverage (2 out of 3 major cerebral artery coverage) and/or do not provide an adequate seal to the cerebral vessels by being unstable in their mechanism of delivery and anchoring. Exemplary assembly 10 and/or device 12 of the present disclosure has at least the following advantages: device 12 provides complete coverage of all three cerebral vessels; provides neuro-deflection with the ability to position and maintain full, unhindered access to cardiac structures for interventional therapies and devices (e.g., reverse condom concept); allows for easy positioning and easy retrieval mechanism; can be fabricated from biocompatible non-thrombogenic material; can include pore size of 100 microns (e.g., to minimize particulate size to the brain); can, in certain embodiments, fit through 7 FR access sheath (smaller than currently designed devices); provides an expanding/collapsing design based on triangulated patterns of flat mesh material—thus manufacturing and scale-up for production is an easy process.

In exemplary embodiments, device 12 can be utilized, without limitation, as an acute procedural implant device 12 to prevent cerebral embolization by providing filter protection to the aortic arch takeoff vessels during: transcatheter aortic valve implantation; atrial fibrillation ablation and left atrial appendage closure.

In some embodiments, device 12 can be utilized, without limitation, as an acute procedural implant device 12 to prevent renal embolization by providing filter protection during the renal arteries during upstream cardiovascular interventional or surgical procedures, including the above procedures.

In certain embodiments, device 12 can be utilized, without limitation, as a short-term implant/device 12 to prevent cerebral embolization during hospitalization after surgical or interventional procedures.

In some embodiments, device 12 (e.g., bioabsorbable variant) can be utilized, without limitation, as a permanent implant to: prevent cerebral embolization with known embolic stroke risk factors or recent surgical or interventional procedures known to increase the risk of stroke; and/or prevent renal embolization in patients with aortic plaque and reduced kidney function.

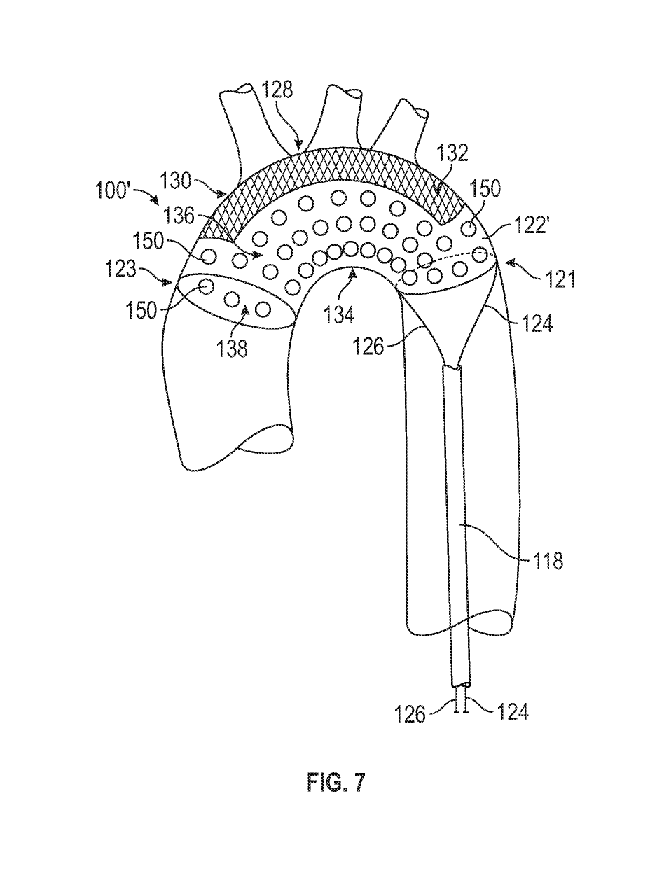

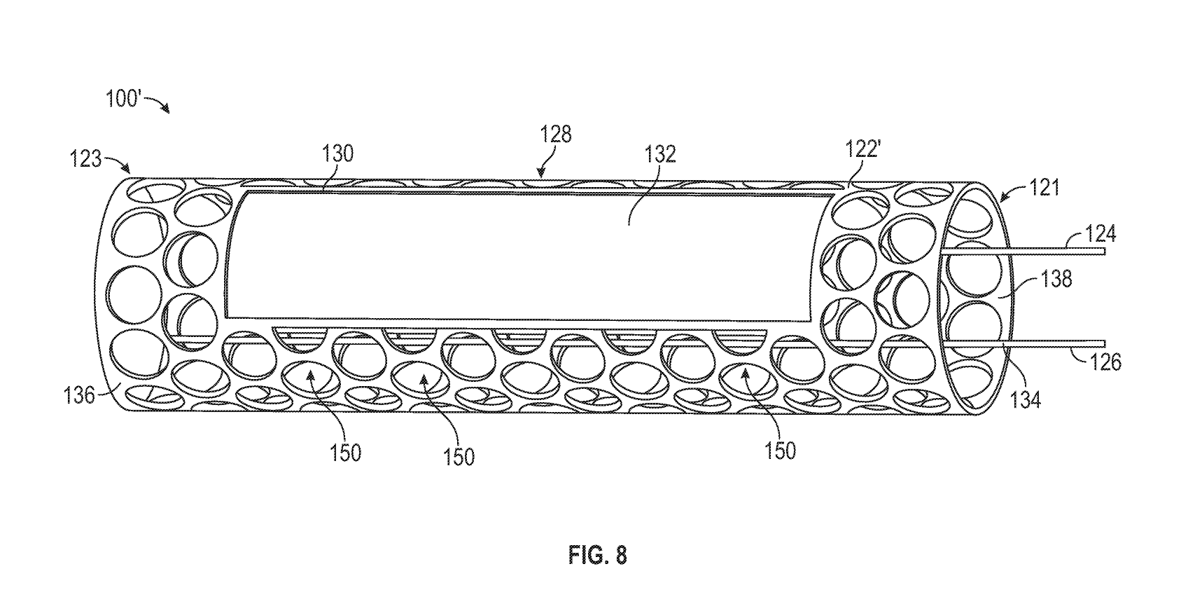

In alternative embodiments and with reference to FIGS. 5 and 6, another exemplary protection assembly 100 (e.g., embolic protection assembly 100) generally includes a bladder member 122, a top tether member 124, a bottom tether member 126, and a delivery or introducer sheath 118. In general, delivery sheath 118 is configured and dimensioned to allow bladder member 122 to be introduced into blood vessels. For example and as shown in FIG. 5, the sheath 118 may be introduced to a desired anatomical location/region, and then bladder member 122 may be introduced to a desired anatomical location/region (e.g., advanced to the aortic arch).

Exemplary bladder member 122 extends from a distal end 121 to a proximal end 123, and a top side 128 of bladder member 122 defines a window portion 130 (e.g., rectangular window portion 130) that is fitted with a porous filter material 132. In general, filter material 132 extends across and covers window portion 130.

In general and as discussed in more detail below, exemplary bladder member 122 is fabricated from an elastic, non-porous material and defines a fully-sealed and continuous bladder member 122. As shown in FIGS. 5 and 6, the bladder member 122 is configured and dimensioned to be delivered in a deflated state (e.g., via sheath 118—FIG. 5), and then aligned within the aortic arch, and then inflated while the clinician uses the top tether member 124 and/or bottom tether member 126 to ensure that the window portion 130 is in line with the arch takeoff vessels (FIG. 6). Inflation and expansion of the bladder member 122 can be actuated via pumping of fluid (e.g., normal saline) into the bladder member 122. For example, the top tether member 124 (and/or bottom tether member 126) can include a fill hose or the like through which fluid is pumped. The bottom tether member 126 can include a support member 134 (e.g., support rod 134) that runs the length of the bladder member 122 and provides longitudinal structure/support to the bladder member 122, even in the deflated state (FIG. 5). FIG. 6 depicts the bladder member 122 in its inflated and expanded state/configuration.

Exemplary bladder member 122 takes the form of a hollow, substantially cylindrical/tubular inflatable bladder member 122. Bladder member 122 can include a number of polymer/elastomer sheets fused and/or bonded together in such a way so as to make a sealed hollow tubular bladder member 122 having an outer wall 136 and an inner wall 138. In general, outer wall 136 and inner wall 138 are sealed together to form an inflatable cavity between the outer and inner walls 136, 138. It is noted that bladder member 122 can be fabricated from a variety of suitable materials.

The inflatable bladder member 122 is configured to be a closed system such that when filled with fluid between the inner wall 138 and the outer wall 136, the bladder member 122 inflates outward to define the inflated hollow tubular bladder member 122 (FIG. 6), with the inflated bladder member 122 capable of maintaining a certain pressure.

The inflatable bladder member 122 can be configured such that when inflated, the outer wall 136 of the bladder member 122 circumferentially apposes the wall of the aorta, thereby creating a seal between the outer wall 136 and the aortic wall such that blood flow cannot substantially pass between them. Stated another way, when the bladder member 122 is expanded from the collapsed position to the expanded position, the outer wall 136 of the expanded bladder member 122 is configured and dimensioned to substantially conform to the aortic wall.

Portions of the outer wall 136 of the inflatable bladder 122 may be texturized (e.g., in regions other than the extreme distal end 123) in order to provide an improved grip between the outer wall 136 and the aortic wall.

As noted above, positioned within the window portion 130 of the inflatable bladder 122 is a porous filter material 132 capable of deflecting and/or blocking the passage of emboli of a certain size while maintaining blood flow to the takeoff vessels (e.g., Brachiocephalic artery, left common carotid artery and left subclavian artery) at the top of the aortic arch.

As shown in FIG. 6, the inflatable bladder member 122 and incorporated filter material 132 are configured and dimensioned in such a way that the bladder member 122 can be positioned such that the filter material 132 covers/protects all three of the takeoff vessels at the top of the aortic arch without impeding blood flow to any of the takeoff vessels, nor impeding downstream blood flow to the descending aorta. In other words, when the bladder member 122 is moved from the collapsed position to the expanded position, the porous filter material 132 is configured and dimensioned to substantially cover the three major cerebral arteries in an aortic arch.

The filter material 132 can be incorporated/mounted into the body of the inflatable bladder 122 in such a way that the bladder member 122 is completely sealed at all of the edges of window 130 shared with the filter material 132 such that the inflatable bladder 122 is a closed system capable of maintaining fluid pressure. Moreover, the filter material 132 can be fused/bonded to the body of the inflatable bladder 122 (e.g., to walls 136 and/or 138) in such a way that when the bladder member 122 is positioned within the aortic arch, only particles of a size smaller than the maximum pore size (e.g., 100 to 150 μm pores) of the filter material 132 are capable of traveling up into the takeoff vessels. Exemplary porous filter material 132 includes a plurality of pores, each pore of the plurality of pores having a pore size of from about 100 μm to about 150 μm.

In exemplary embodiments, both the materials/components of the inflatable bladder 122 and the filter material 132 are of a flexible/malleable nature such that the bladder member 122 and incorporated filter material 132 are capable of expanding and contracting and accurately contouring to the shape of the aortic arch.

In exemplary embodiments and as shown in FIGS. 5 and 6, the bottom tether member 126 can include a tether or wire member 126 that is attached to and extends from the distal end 121 of the bladder member 122, into the delivery sheath 118 and out through the entry point of the patient.

The support member or support rod 134 can include a semi-rigid rod or wire 134 that is attached to the bottom tether member 126 at the distal end 121, or can be a continuation/extension of the bottom tether member 126 at and from the distal end 121. In exemplary embodiments, support member 134 extends along the bottom length of the bladder member 122, as shown in FIGS. 5 and 6.

In general, the support member 134 (e.g., semi-rigid rod or wire 134) is attached to the inflatable bladder 122 in such a way that it does not disrupt the closed system of the inflatable bladder 122 nor prevent it from maintaining pressure. For example and as shown in FIG. 5, support member 134 can be attached to bladder member 122 at the distal end 121 and the proximal end 123 of bladder member 122. As shown in FIG. 5, support member 134 is attached to outer wall 136 at the distal end 121 and the proximal end 123 of bladder member 122.

In other embodiments, it is noted that support member 134 can be positioned within inner wall 138 and attached to inner wall 138 of bladder member 122 (e.g., at the distal end 121 and the proximal end 123 of bladder member 122). In further embodiments, support member 134 can be positioned between inner wall 138 and outer wall 136, and attached to inner wall 138 and/or outer wall 136.

In general, support member 134 provides longitudinal structure and support to the bladder member 122 both in an expanded/inflated state and a collapsed/deflated state (FIGS. 5 and 6). In some embodiments, the support member 134 is fabricated from a shape memory alloy (e.g., nitinol) or shape memory polymer or the like, although the present disclosure is not limited thereto. Rather, it is noted that support member 134 can be fabricated from a variety of suitable materials, and can take a variety of shapes/forms.

The top tether or wire member 124 can be connected or attached to the top side 128 of bladder member 122 at the distal end 121 of bladder member 122.

The top tether member 124 extends from the distal end 121, into the delivery sheath 118, and out through the entry point of the patient. In exemplary embodiments, top tether member 124 includes or is associated with a fill hose or the like, through which fluid may be pumped/moved in order to fill/inflate or empty/deflate the inflatable/deflatable bladder member 122.

The top tether member 124 at its end outside of the patient can be connected to a syringe or other device capable of holding and pumping fluid. The top tether member 124 can also be connected at its end outside of the patient to a pressure gauge that is capable of measuring the fluid pressure within the inflatable bladder member 122. In general, the top tether member 124 is capable of being sealed at its end outside of the patient in order to create a closed system so that fluid pressure can be maintained within the bladder member 122.

The top tether member 124 and/or bottom tether member 126 may be manipulated by the physician/surgeon to properly position the bladder member 122 within the aortic arch.

For example, the top and/or bottom tether members 124, 126 may be manipulated to advance the bladder member 122 within the delivery sheath 118, advance the bladder member 122 out of the delivery sheath 118, advance the bladder member 122 within the aorta into position, rotate the bladder member 122 into alignment within the aortic arch, and/or retract the bladder member 122 back into the delivery sheath 118 and back outside of the patient.

FIG. 5 depicts the bladder member 122 in its collapsed/deflated state. Bladder member 122 has a significantly smaller outer diameter in its collapsed/deflated state (FIG. 5) than in its expanded/inflated state (FIG. 6). FIG. 5 depicts the state of the bladder member 122 as it would be within the delivery sheath 118, immediately after advancement out of the delivery sheath 118, and immediately before retrieval back into the delivery sheath 118. In general, the bladder member 122 is fabricated from materials of a flexible/malleable nature such that the integrity of the bladder member 122 is not compromised when the materials are compacted/compressed into the state depicted in FIG. 5.

In certain embodiments and as noted, the support member 134 (e.g., semi-rigid rod/wire 134) is attached to the bladder member 122 at its distal and proximal ends 121, 123. In some embodiments and as shown in FIG. 5, when the bladder member 122 is in the deflated state/configuration, the support member 134 may be capable of physically separating from the body of the bladder member 122 (e.g., from the outer wall 136) at the points along the bladder member 122 (e.g., along the bottom side of the bladder member 122 and away from outer wall 136) except for the extreme attached distal and proximal ends 121, 123. Exemplary support member 134 is configured and dimensioned to maintain its shape even in the compressed/deflated state of the bladder member 122. As such, the support member 134 can provide longitudinal structural integrity to the bladder member 122, even in the compressed/deflated state, such that manipulating the bottom tether member 126 applies force across the length of the support member 134 (e.g., from attached end 121 to attached end 123) capable of moving the bladder member 122 into position.

In exemplary embodiments, various members/structures of assembly 100 may contain or include certain radio opaque elements, including without limitation the support member 134 and top and bottom tether members 124, 126, such that the physician/surgeon can use fluoroscopy to determine the position of the bladder member 122 within the aortic arch.

Once the compressed/deflated bladder member 122 is in the proper position within or adjacent to the aortic arch (e.g., with the filter material 132 facing up towards the takeoff vessels and the support member 134 positioned towards the bottom of the aortic arch) the physician/surgeon may use the fill hose of top tether member 124 to pump fluid into the inflatable bladder member 122 (e.g., between walls 136, 138) and begin to expand/inflate the bladder member 122.

As noted, assembly 100 may include certain radio opaque elements such that the physician/surgeon is able to use fluoroscopy while inflating/expanding the bladder member 122 to ensure that the bladder member 122 remains in proper alignment. For example, if for some reason the bladder member 122 is not properly aligned, or the bladder member 122 needs to be removed, the physician/surgeon may use the hose of the top tether member 124 to deflate the bladder member 122.

When the procedure is complete, or when the physician/surgeon wishes to remove the bladder member 122, the bladder member 122 may be deflated by applying negative pressure to the hose of the top tether member 124 until the bladder member 122 reaches its (fully) compressed/deflated state, as depicted in FIG. 5.

Once in its (fully) compressed/deflated state, the physician/surgeon may use the top and/or bottom tether members 124, 126 to retract the bladder member 122 back into the delivery sheath 118 and out of the patient.

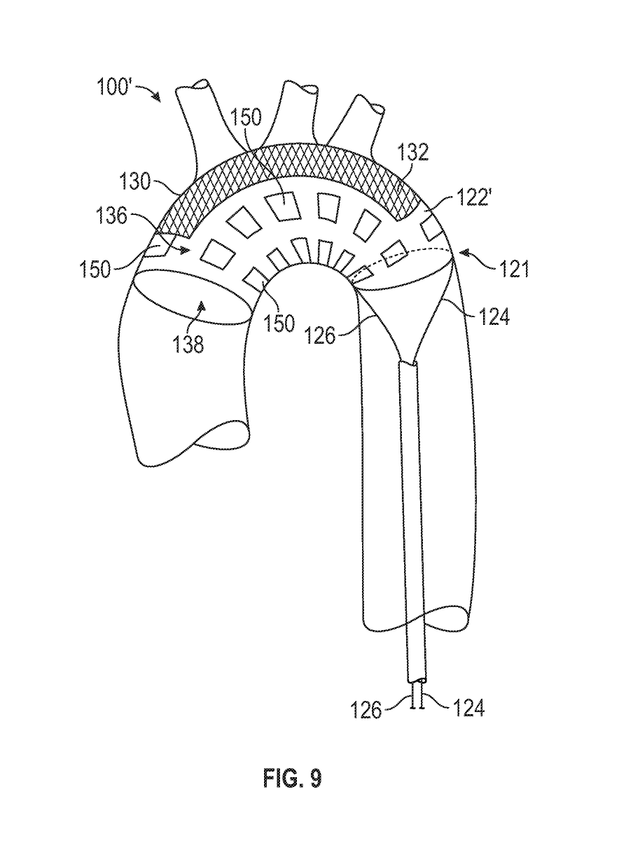

FIGS. 7 and 8 depict another embodiment of a protection assembly 100′ showing the bladder member 122′ in its expanded/inflated state.

In this embodiment, bladder member 122′ of assembly 100′ functions similar to bladder member 122 of assembly 100 discussed above, except that bladder member 122′ includes a plurality of perforations or apertures 150 through bladder member 122′ (e.g., through walls 136, 138 of bladder member 122′).

In exemplary embodiments, the perforations/apertures 150 are configured and dimensioned such that the edges of the inflatable bladder member 122′ (e.g., edges of walls 136, 138) surrounding the perforations 150 are sealed, making the inflatable bladder member 122′ a closed system capable of maintaining fluid pressure, similar to bladder member 122 without apertures 150.

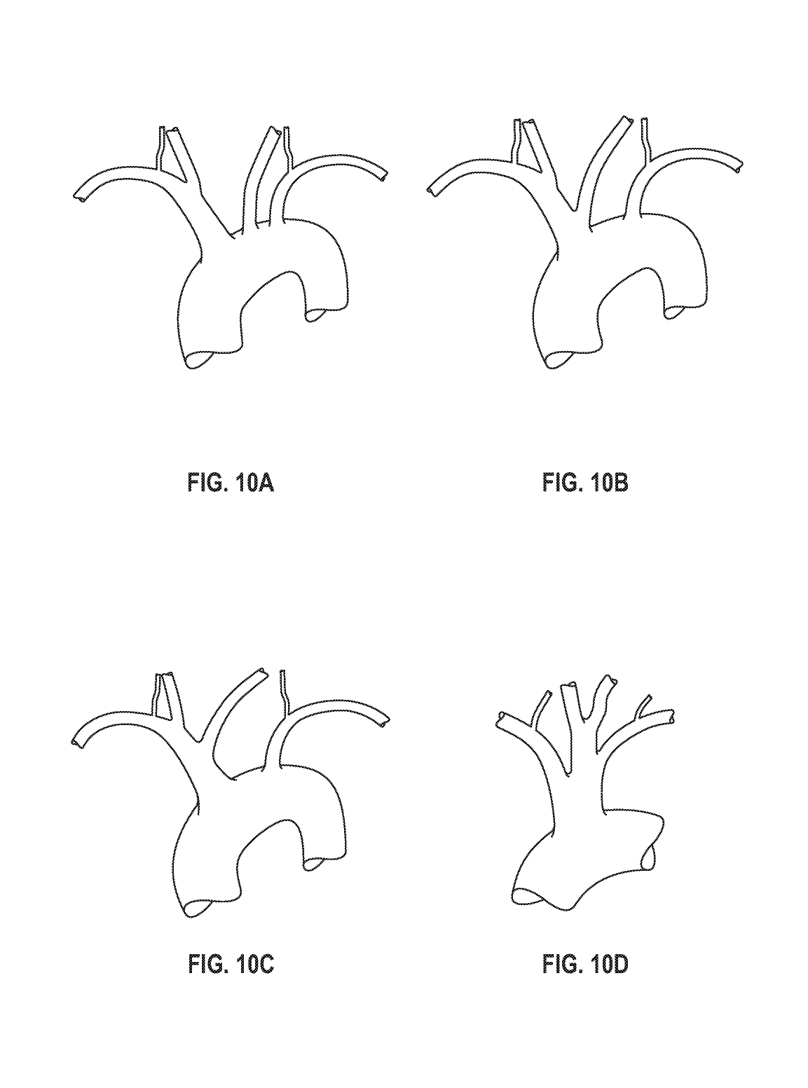

Exemplary perforations or apertures 150 may be elliptical or circular in shape, as depicted in FIGS. 7 and 8, although the present disclosure is not limited thereto. In another embodiment and as shown in FIG. 9, perforations or apertures 150 may be quadrilateral or square in shape.

It is noted that other shapes than an elliptical/circular shape or quadrilateral/square shape may be provided for perforations/apertures 150. For example, the perforations/apertures 150 may define other shapes (e.g., polygonal shapes such as tetragons, pentagons, heptagons, octagons, etc., and/or regular or irregular shapes, rhombi, etc., or combinations thereof).

The perforations 150 may be of a size such that the volume of fluid required to fully inflate the bladder member 122′ is minimized without compromising the structural integrity of the inflatable bladder member 122′.

In exemplary embodiments, the edges of the perforations 150 provide anchor points between the outer and inner walls 136, 138 of the inflatable bladder member 122′, thereby decreasing the volume of fluid required to inflate the bladder member 122′.

The size of the perforations 150 may be configured and dimensioned such that the thickness of the inflated bladder 122′ (the distance between the outer and inner walls 136, 138 of the bladder when fully inflated) is a desired thickness.

In general, the perforations can serve as a safety mechanism for the bladder member 122′ if it is aligned improperly within the aortic arch and inflated fully. For example, the perforations 150 can prevent full occlusion of any of the takeoff vessels if the bladder member 122′ is misaligned and fully inflated such that the body or wall 136 of the inflatable bladder 122′ covers the top of the aortic arch rather than the filter region 132 covering the top of the aortic arch. In other words, respectively positioned perforations 150 will allow fluid flow through takeoff vessels if the perforations 150 are positioned under the takeoff vessels.

It is noted that perforations 150 may be present throughout the entire body or walls 136, 138 of the inflatable bladder member 122′, or only in areas of the inflatable bladder member 122′ immediately surrounding the filter region/material 132.

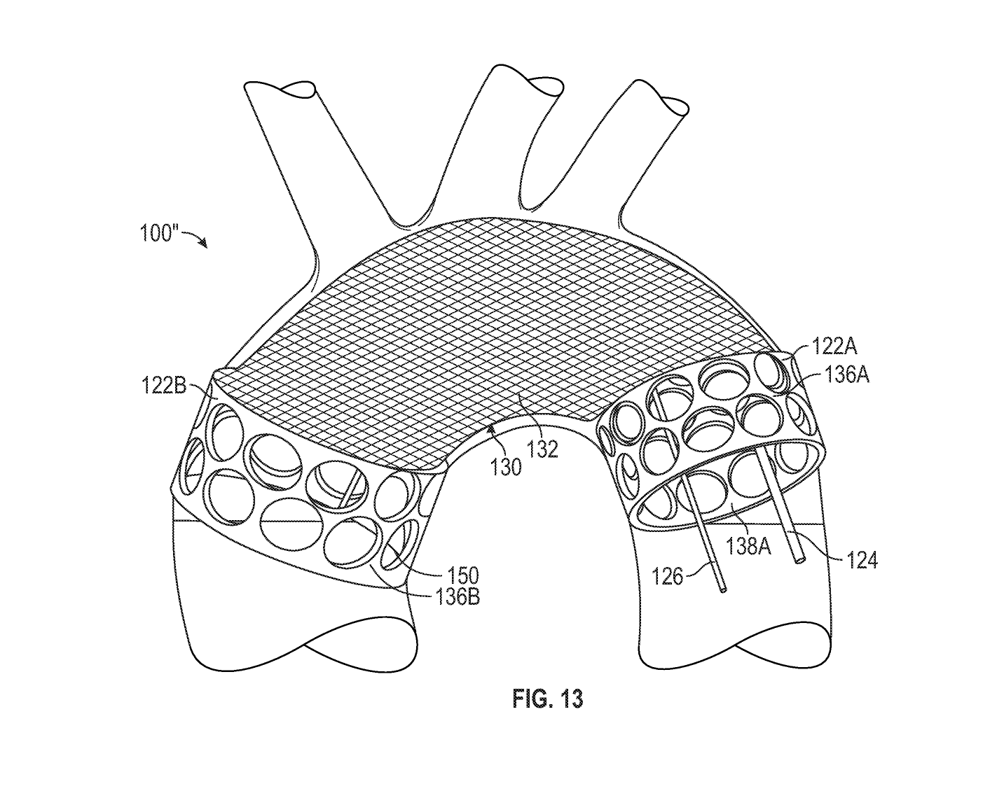

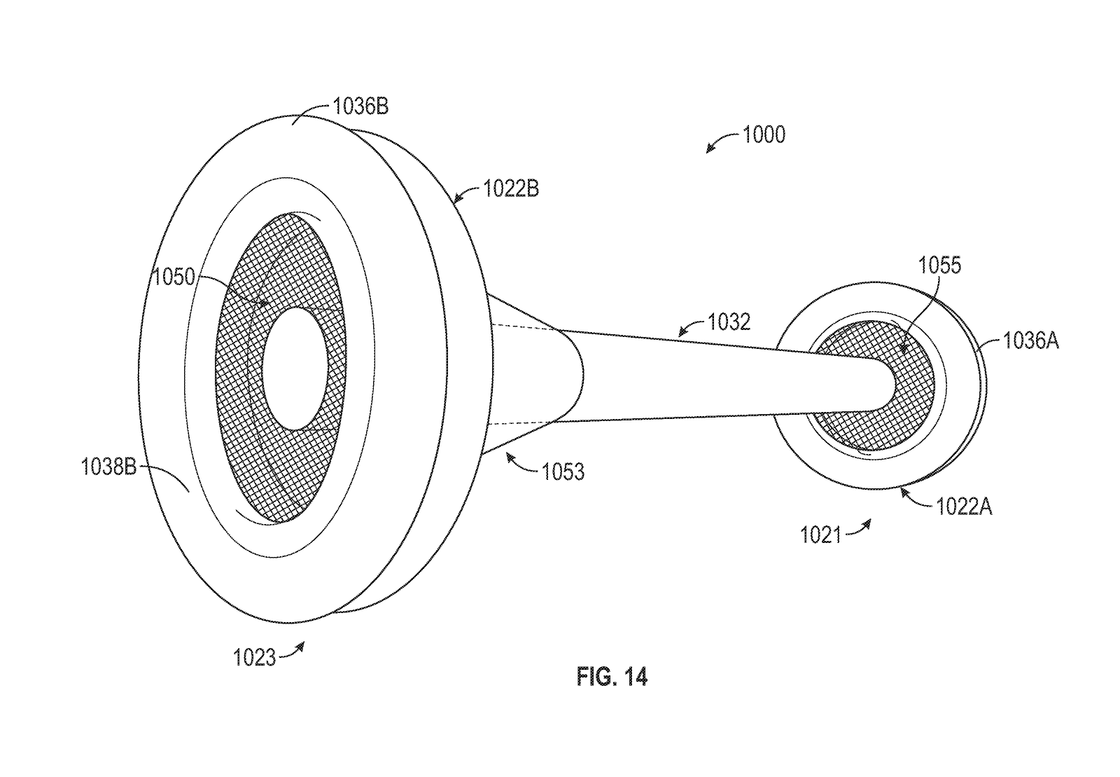

FIGS. 12 and 13 depict another embodiment of a protection assembly 100″.

In this embodiment, first bladder member 122A and second bladder member 122B of assembly 100″ function similar to bladder member 122 or 122′ discussed above. First bladder member 122A and/or second bladder member 122B can include a plurality of perforations or apertures 150 through bladder member 122A and/or 122B (e.g., through walls 136A, 138A of bladder member 122A, and/or through walls 136B, 138B of bladder member 122B).

In exemplary embodiments, first bladder member 122A is positioned at distal end 121, and second bladder member 122B is positioned at proximal end 123, with window portion 130 positioned between members 122A, 122B.

In exemplary embodiments, bladder members 122A, 122B take the form of inflatable/deflatable cylindrical support members 122A, 122B. Exemplary window portion 130 includes hollow cylindrical member 132 (e.g., cylindrical filter material 132) that takes the form of a hollow cylindrical window region 130.

First bladder member 122A includes outer wall 136A and inner wall 138A that are sealed together such that member 122A is configured to hold fluid pressure. Second bladder member 122B includes outer wall 136B and inner wall 138B that are sealed together such that member 122B is configured to hold fluid pressure.

Inflation and expansion of the bladder member 122A can be actuated via pumping of fluid (e.g., normal saline) into the bladder member 122A at one or more points. For example, the top tether member 124 (and/or bottom tether member 126) can include a fill hose or the like through which fluid is pumped. The bottom tether member 126 (and/or top tether member 124) can include a support member 134 (e.g., support rod 134) that runs at least the length of the bladder member 122A and provides longitudinal structure/support to the bladder member 122A.

Inflation and expansion of the bladder member 122B can be actuated via pumping of fluid (e.g., normal saline) into the bladder member 122B at one or more points. For example, the bottom tether member 126 (and/or top tether member 124) can include a fill hose or the like through which fluid is pumped. The bottom tether member 126 (and/or top tether member 124) can include a support member 134 that runs the length of the bladder member 122A, the filter material 132 and/or the second bladder member 122B, and provides longitudinal structure/support to the bladder member 122A, filter material 132 and/or bladder member 122B.

Tether members 124 and/or 126 can be utilized by a user to position assembly 100″ as desired (e.g., within a patient). Tether members 124 and/or 126 can be utilized by a user to expel and retrieve assembly 100″ from delivery sheath 118. Tether members 124 and/or 126 can be utilized by a user to rotate and/or position assembly 100″ as desired (e.g., within a patient). Tether members 124 and/or 126 can provide longitudinal structural rigidity to assembly 100″. Tether members 124 and/or 126 can include a shape memory alloy/polymer that facilitates actuation of assembly 100″.

In certain embodiments, cylindrical member 132 (e.g., cylindrical filter material 132) extends from first member 122A to second member 122B. Filter material 132 (e.g., cylindrical filter material region 132) can include a metal and/or polymer mesh material 132. Filter material 132 can include a plurality of pores large enough to allow for the flow of blood, and small enough to deflect clinically significant emboli. Exemplary porous filter material 132 includes a plurality of pores, each pore of the plurality of pores having a pore size of from about 100 μm to about 150 μm.

The distal end 121 of member/filter material 132 is attached circumferentially to member 122A, and the proximal end 123 of member/filter material 132 is attached circumferentially to member 122B. As such, when members 122A, 122B are inflated, the cylindrical and hollow filter material/member 132 is in the open position (FIG. 13). In general, open filter material/member 132 is configured to extend the length of the aortic arch, and is capable of deflecting emboli from entering takeoff vessels of the aortic arch.

In exemplary embodiments, after the bladder members 122A, 122B are positioned in a desired anatomical location, introduction of fluid to the bladder members 122A, 122B causes the bladder members 122A, 122B to expand from a collapsed position to an expanded position.

After the bladder members 122A, 122B are positioned in the expanded position, removal of fluid from the bladder members 122A, 122B causes the bladder members 122A, 122B to collapse from the expanded position to the collapsed position.

When the bladder members 122A, 122B are expanded from the collapsed position to the expanded position, the outer walls 136A, 136B of the expanded bladder members 122A, 122B are configured and dimensioned to substantially conform to an aortic wall of the desired anatomical location.

When the bladder members 122A, 122B are moved from the collapsed position to the expanded position, the porous filter material 132 is configured and dimensioned to substantially cover three major cerebral arteries in an aortic arch of the desired anatomical location.

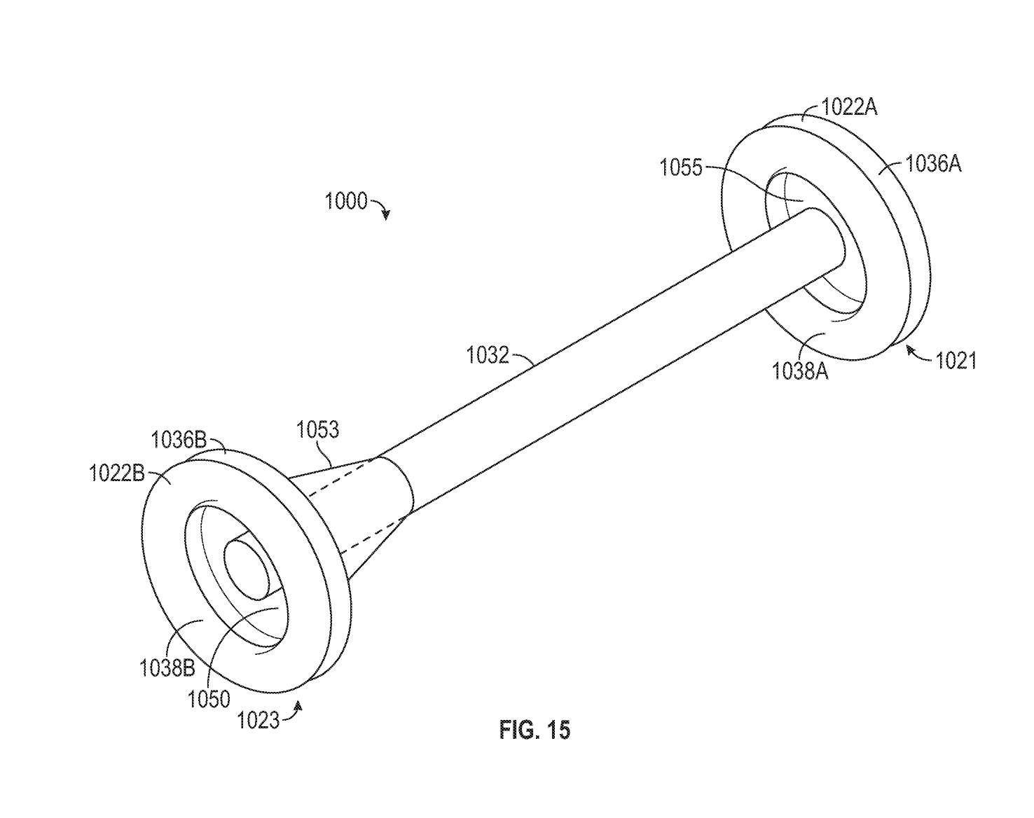

FIGS. 14-16 depict another embodiment of a protection assembly 1000.

In this embodiment, first bladder member 1022A and second bladder member 1022B of assembly 1000 function similar to bladder member 122 or 122′ or 122″ discussed above. First bladder member 1022A and/or second bladder member 1022B may or may not include a plurality of perforations or apertures (e.g., apertures 150) through bladder member 1022A and/or 1022B (e.g., through walls 1036A, 1038A of bladder member 1022A, and/or through walls 1036B, 1038B of bladder member 1022B).

In exemplary embodiments, first bladder member 1022A is positioned at distal end 1021, and second bladder member 1022B is positioned at proximal end 1023, with hollow cylindrical member 1032 positioned between and mounted with respect to members 1022A, 1022B.

In exemplary embodiments, bladder members 1022A, 1022B take the form of inflatable/deflatable cylindrical/toroidal support members 1022A, 1022B.

First bladder member 1022A includes outer wall 1036A and inner wall 1038A that are sealed together such that member 1022A is configured to hold fluid pressure. Second bladder member 1022B includes outer wall 1036B and inner wall 1038B that are sealed together such that member 1022B is configured to hold fluid pressure.

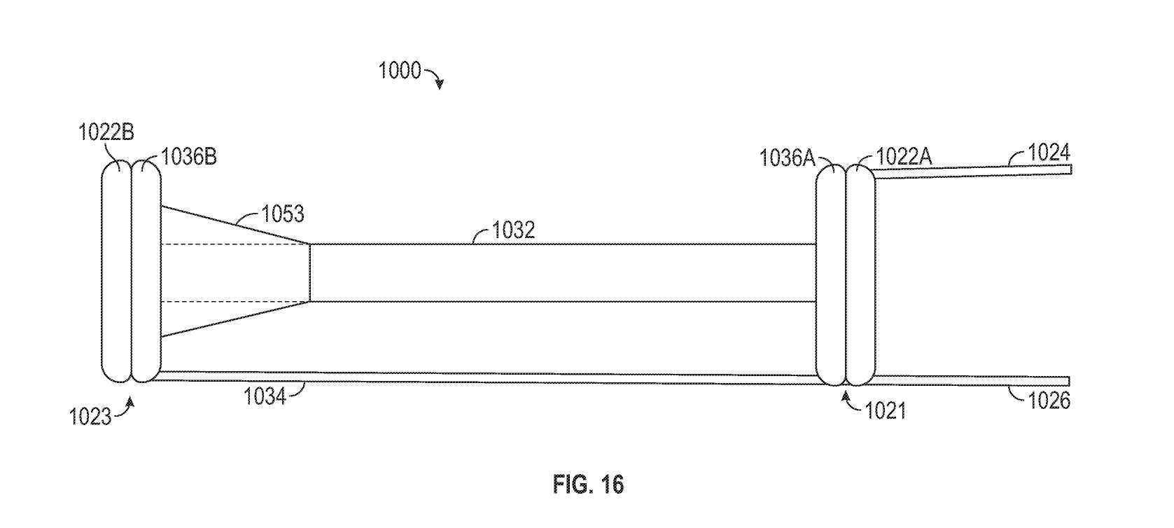

Inflation and expansion of the bladder member 1022A can be actuated via pumping of fluid (e.g., normal saline) into the bladder member 1022A at one or more points. For example and as shown in FIG. 16, the top tether member 1024 (and/or bottom tether member 1026) can include a fill hose or the like through which fluid is pumped. The bottom tether member 1026 (and/or top tether member 1024) can include a support member 1034 (e.g., support rod 1034) that runs at least the length of the bladder member 1022A and provides longitudinal structure/support to the bladder member 1022A.

Inflation and expansion of the bladder member 1022B can be actuated via pumping of fluid (e.g., normal saline) into the bladder member 1022B at one or more points. For example, the bottom tether member 1026 (and/or top tether member 1024) can include a fill hose or the like through which fluid is pumped. The bottom tether member 1026 (and/or top tether member 1024) can include a support member 1034 that runs the length of the bladder member 1022A, the cylindrical member 1032 and/or the second bladder member 1022B, and provides longitudinal structure/support to the bladder member 1022A, member 1032 and/or bladder member 1022B.

Tether members 1024 and/or 1026 can be utilized by a user to position assembly 1000 as desired (e.g., within a patient). Tether members 1024 and/or 1026 can be utilized by a user to expel and retrieve assembly 1000 from delivery sheath 118. Tether members 1024 and/or 1026 can be utilized by a user to rotate and/or position assembly 1000 as desired (e.g., within a patient). Tether members 1024 and/or 1026 can provide longitudinal structural rigidity to assembly 1000. Tether members 1024 and/or 1026 can include a shape memory alloy/polymer that facilitates actuation of assembly 1000.

In certain embodiments, hollow cylindrical member 1032 extends from first member 1022A to second member 1022B, and is mounted with respect to members 1022A, 1022B, as discussed further below (e.g., mounted to first and second proximal filter members 1050, 1053, and mounted to distal filter member 1055).

Cylindrical member 1032 can be non-porous, or it can be porous. In general, member 1032 is configured to allow for the flow of blood across the aortic arch, and is configured to allow for the passage of surgical/procedural equipment across the aortic arch.

In certain embodiments, cylindrical member 1032 is non-porous, and takes the form of a non-porous plastic and/or polymer material 1032. Such non-porous plastic and/or polymer material of member 1032 can be flexible and configured of contouring with the curvature of the aortic arch or the like.

In other embodiments, cylindrical member 1032 is porous, and includes a metal mesh and/or plastic polymer mesh material 1032. Such porous plastic and/or polymer material of member 1032 can be flexible and configured of contouring with the curvature of the aortic arch or the like.

Porous filter material/member 1032 can include a plurality of pores large enough to allow for the flow of blood, and small enough to deflect clinically significant emboli.

In certain embodiments and as discussed further below, the distal end 1021 of member 1032 is attached circumferentially to an inner diameter of distal filter member 1055, and the proximal end 1023 of member 1032 is attached circumferentially to an inner diameter of first and second proximal filter members 1050, 1053.

In exemplary embodiments, first proximal filter member 1050 (e.g., toroidal filter member 1050) is mounted to bladder member 1022B and to cylindrical member 1032.

More particularly, first proximal filter member 1050 is attached along its outer and larger circumference to bladder member 1022B, and is attached along its inner and smaller circumference to the extreme proximal end 1023 of member 1032.

Exemplary first proximal filter member 1050 includes a metal and/or polymer mesh material 1050, and can include a plurality of pores large enough to allow for the flow of blood, and small enough to deflect clinically significant emboli. Exemplary filter member 1050 is configured to trap emboli of a size larger than that of the filter pore size from travelling downstream/distally.

In exemplary embodiments, second proximal filter member 1053 (e.g., conical filter member 1053) is mounted to bladder member 1022B and to cylindrical member 1032.

More particularly, second proximal filter member 1053 is attached along its outer and larger circumference to bladder member 1022B, and is attached along its inner and smaller circumference to a position along the proximal end 1023 of member 1032.

Exemplary first proximal filter member 1053 includes a metal and/or polymer mesh material 1053, and can include a plurality of pores large enough to allow for the flow of blood, and small enough to deflect clinically significant emboli. Exemplary filter member 1053 is configured to trap emboli of a size larger than that of the filter pore size from travelling downstream/distally.

In exemplary embodiments, distal filter member 1055 (e.g., toroidal filter member 1055) is mounted to bladder member 1022A and to cylindrical member 1032.

More particularly, distal filter member 1055 is attached along its outer and larger circumference to bladder member 1022A, and is attached along its inner and smaller circumference to the extreme distal end 1023 of member 1032.

Exemplary distal filter member 1055 includes a metal and/or polymer mesh material 1055, and can include a plurality of pores large enough to allow for the flow of blood, and small enough to deflect clinically significant emboli. Exemplary filter member 1055 is configured to trap emboli of a size larger than that of the filter pore size from travelling downstream/distally.

It is noted that distal filter 1055 can take the form of filter 1053 (e.g., conical), and be attached along its outer and larger circumference to bladder member 1022A, and be attached along its inner and smaller circumference to the a position along the distal end 1021 of member 1032. It is also noted that distal end 1021 can include first and second filters, similar to filters 1050, 1053 of proximal end. Other suitable combinations and permutations of filters 1050, 1053, 1055 can be provided to assembly 1000.

In exemplary embodiments, after the bladder members 1022A, 1022B are positioned in a desired anatomical location, introduction of fluid to the bladder members 1022A, 1022B causes the bladder members 1022A, 1022B to expand from a collapsed position to an expanded position.

After the bladder members 1022A, 1022B are positioned in the expanded position, removal of fluid from the bladder members 1022A, 1022B causes the bladder members 1022A, 1022B to collapse from the expanded position to the collapsed position.

When the bladder members 1022A, 1022B are expanded from the collapsed position to the expanded position, the outer walls 1036A, 1036B of the expanded bladder members 1022A, 1022B are configured and dimensioned to substantially conform to an aortic wall of the desired anatomical location.

When the bladder members 1022A, 1022B are moved from the collapsed position to the expanded position, the cylindrical member 1032 is configured and dimensioned to allow for the flow of blood across an aortic arch of the desired anatomical location.

The present disclosure will be further described with respect to the following example; however, the scope of the disclosure is not limited thereby. The following example illustrates the advantageous protection assemblies, devices and methods of the present disclosure.

### Example 1: Design Specifications—Aortic Arch Embolic Protection Device

One purpose of Example 1 is to outline the design input specifications/requirements (clinical and performance) to guide development of exemplary aortic sleeve filter device 12.

It is noted that one relevant standard is FDA CDRH 1658:2008, Guidance for Industry and FDA Staff: Coronary and Carotid Embolic Protection Devices—Premarket Notification [510 (k)] Submissions.

### Access and Delivery:

In its initial compressed/collapsed state, exemplary device 12 will fit within a 7 French catheter or less (about 2.3 mm OD; diameter [mm]=Fr/3) for percutaneous introduction into the femoral artery.

Device 12 can be supplied with the equipment necessary to insert the device 12 into an off-the-shelf delivery sheath 16, or pre-mounted on the delivery system 16. It is noted that ease of preparation is important.

Once in the delivery catheter 16 or the like, the device 12 will be navigable through the vasculature to the aortic arch, without permanent deformation of the device 12.

### Deployment and Positioning:

Once in the aortic arch, the catheter 16 can be withdrawn. The device 12 margins (at minimum) can be visualizable on fluoroscopy to allow proper positioning.

When the device 12 is in position, it can be expandable to appose the circumference of the aortic arch (e.g., within the parameters set under “Coverage” below) without damaging the vessel wall. The device 12 can maintain position once placed (resisting migration due to blood flow or aspects of the index procedure). The device 12 can be collapsible and re-expandable to allow for re-positioning and redeployment if necessary.

### Coverage:

When expanded, the device can be able to provide coverage of all aortic arch branch vessels, independent of:

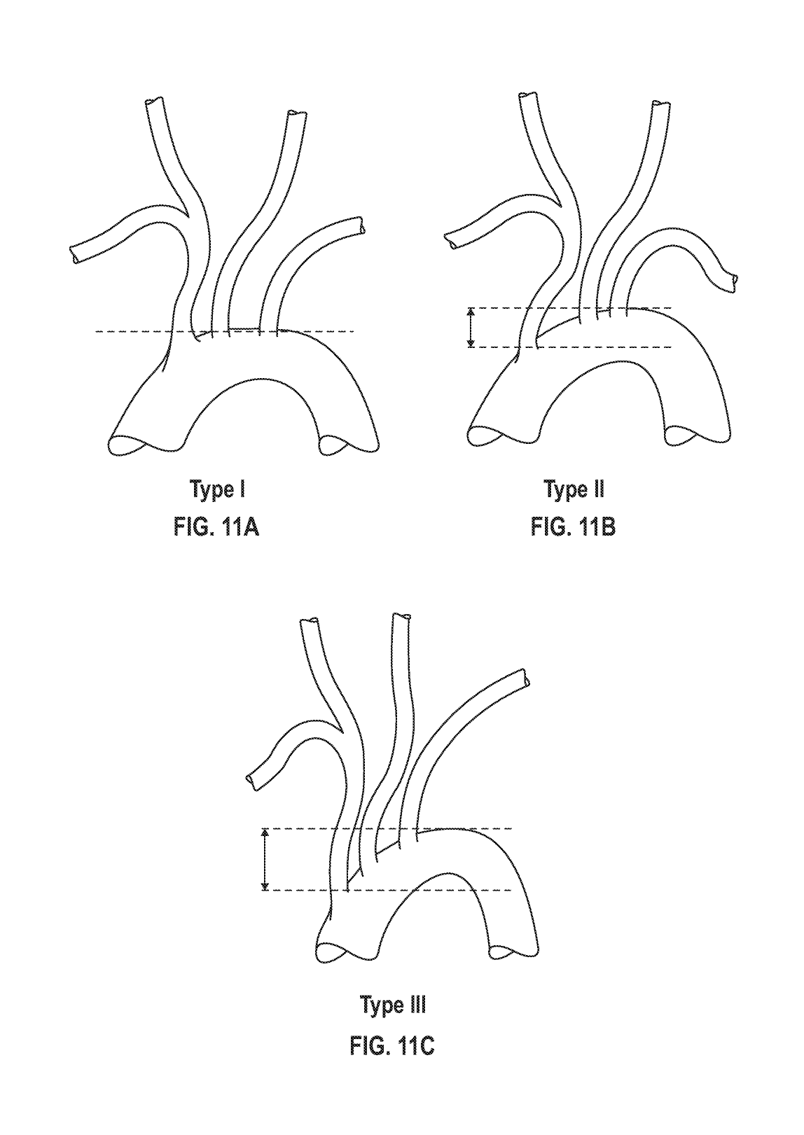

(i) aortic arch anatomic type (see FIGS. 10A-10D for various aortic arch anatomic types);

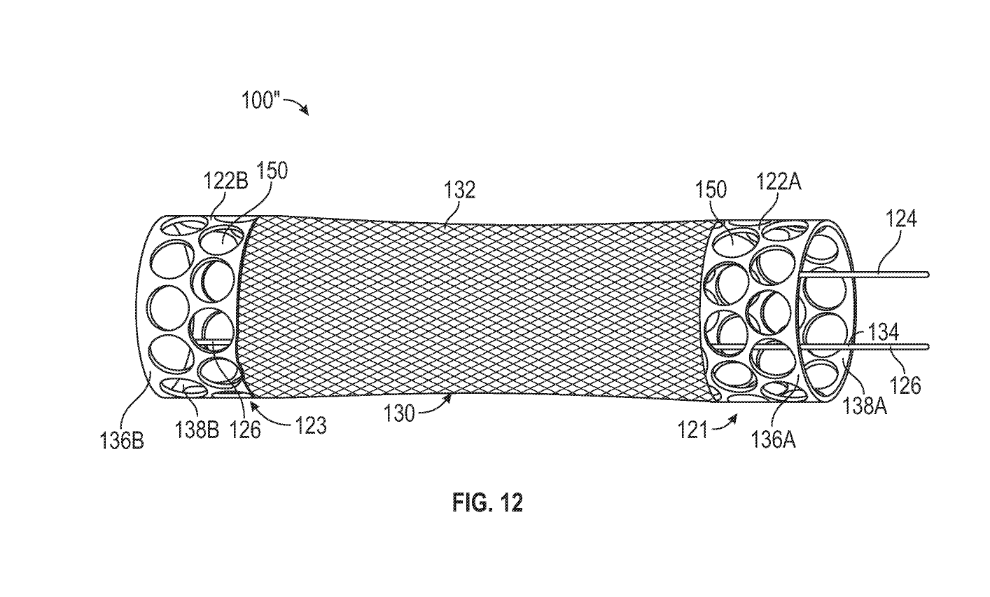

(ii) cerebral vessel takeoff positioning (see FIGS. 11A-11C for various cerebral vessel takeoff positioning types);

(iii) aortic arch diameter (height): estimated range 23-41 mm. BSA-adjusted ascending aorta 1.4-2.1 cm/m² body surface area, absolute upper bound 3.8 cm. Adult BSA 95% CIs for women and men respectively range from 1.70-1.92, corresponding to aortic diameters of 23-41 mm (excluding descending aorta); see also Table 1 below;

(iv) Coverage Length (Most proximal to most distal ostium)

TABLE 1
Normal aortic dimensions in adults

| Diameter | | | |
|---|---|---|---|
| Aortic annulus | | | |
| Male | 2 · 6 ± 0 · 3 | cm | TTE^{[33]} |
| Female | 2 · 3 ± 0 · 2 | cm | TTE^{[33]} |
| Sinus of Valsalva | | | |
| Male | 3 · 4 ± 0 · 3 | cm | TTE^{[33]} |
| Female | 3 · 0 ± 0 · 3 | cm | TTE^{[33]} |
| Aortic root | <3 · 7 | cm | TTE^{[33]} |
| Proximal ascending aorta | | | |
| Male | 2 · 9 ± 0 · 3 | cm | TTE^{[33]} |
| Female | 2 · 6 ± 0 · 3 | cm | TTE^{[33]} |
| Ascending aorta | 1 · 4-2 · 1 | cm · m⁻² | TEE^{[45]} |
| | <3 · 8 | cm (2 · 5-3 · 8) | CT^{[2]} |
| | <3 · 7 | cm | TTE^{[46]} |
| Descending aorta | 1 · 0-1 · 6 | cm · m⁻² | TEE^{[45]} |
| | <2 · 8 | cm (1 · 7-2 · 8) | CT^{[2]} |
| Wall thickness | | | |
| Aortic wall | <4 | mm | CT^{[47]} |
| | <3 | mm | Angio^{[48]} |
| | <4 | mm | TEE^{[49]} |

### Device Interaction:

Once deployed, the device 12 will not interfere with the index procedure. In particular, it will allow advancement and withdrawal of the index procedure guide wires and catheters without: interference with the manipulation of catheters or wires; damage to catheters, wires or other devices; experiencing loss of positioning or loss of filter integrity; obstruction to cerebral blood flow; damage to the vessel wall (e.g., by proximal or distal device edge or folds).

### Dwell Time:

In exemplary embodiments, device 12 will be capable of remaining in the body for greater than or equal to about 4 hours.

### Filter:

When in an expanded state (e.g., for the range of arch diameters expected), the exemplary device 12 will: allow adequate perfusion to the brain; prevent the passage of particles greater than or equal to about 100 μm; prevent the passage of blood and particles between the filter device 12 and the aortic wall; and avoid entrapment of particles and/or occlusion of filter pores.

### Retrieval:

At the end of the index procedure, the device 12 will be able to be collapsed and retrieved into the delivery sheath 16 for removal from the body without the release of embolic material and without damage to the vessel wall.

### Safety Requirements:

Exemplary device 12 can be biocompatible (e.g., non-cytotoxic, non-sensitizing, non-hemolytic, thromboresistant, immune compatible, non-pyrogenic); can provide adequate cerebral perfusion (high porosity)—pressure gradient/flow rate reduction less than or equal to 10-15%; and provide substantially no damage to the vascular endothelium during expansion, indwelling, or retrieval.

### Manufacturing Requirements:

Exemplary device 12 can be sterilizable (single use); scalable to mass production; have a shelf life greater or equal to about 6 months; and be stable through transportation environmental conditions.

### FDA Classification:

| Device | |
|---|---|
| Regulation Description | Percutaneous catheter OR |
| | Cardiovascular intravascular filter |
| Regulation | Cardiovascular |
| Medical Specialty | |
| Review Panel | Cardiovascular |
| Product Code | Office of Device Evaluation (ODE) |
| | Division of Cardiovascular Devices (DCD) |
| | Interventional Cardiology Devices Branch |
| | (ICDB)/Peripheral Interventional Devices |
| | Branch/Structural Heart Device Branch |
| Premarket Review | |
| Submission Type | 510k |
| Regulation Number | §870.1250 OR §870.3375 |
| Device Class | 2/3 |

### CE Classification:

| | |
|---|---|
| Category | External communicating device |
| Contact | Blood |
| Contact Duration | A - Limited (≤24 h) |
| CE Classification | Class III |

Whereas the disclosure has been described in connection with protection assemblies/devices for cardiac or endovascular procedures/applications, such description has been utilized only for purposes of disclosure and is not intended as limiting the disclosure. To the contrary, it is to be recognized that the disclosed protection assemblies/devices and related instruments/systems are capable of use for other procedures/applications (e.g., protection assemblies/devices for orthopedic procedures/applications or other surgical procedures/applications).

Although the systems/methods of the present disclosure have been described with reference to exemplary embodiments thereof, the present disclosure is not limited to such exemplary embodiments/implementations. Rather, the systems/methods of the present disclosure are susceptible to many implementations and applications, as will be readily apparent to persons skilled in the art from the disclosure hereof. The present disclosure expressly encompasses such modifications, enhancements and/or variations of the disclosed embodiments. Since many changes could be made in the above construction and many widely different embodiments of this disclosure could be made without departing from the scope thereof, it is intended that all matter contained in the drawings and specification shall be interpreted as illustrative and not in a limiting sense. Additional modifications, changes, and substitutions are intended in the foregoing disclosure. Accordingly, it is appropriate that the appended claims be construed broadly and in a manner consistent with the scope of the disclosure.

1. A protection assembly comprising:
a bladder member extending from a proximal end to a distal end, the bladder member having an outer wall and an inner wall sealed together to form an inflatable cavity between the outer and inner walls, the bladder member defining a window portion;
a porous filter material mounted to the bladder member, the porous filter material extending across and covering the window portion;
wherein after the bladder member is positioned in a desired anatomical location, introduction of fluid to the bladder member causes the bladder member to expand from a collapsed position to an expanded position.

2. The assembly of claim 1, wherein after the bladder member is positioned in the expanded position, removal of fluid from the bladder member causes the bladder member to collapse from the expanded position to the collapsed position.

3. The assembly of claim 1, wherein when the bladder member is expanded from the collapsed position to the expanded position, the outer wall of the expanded bladder member is configured and dimensioned to substantially conform to an aortic wall of the desired anatomical location.

4. The assembly of claim 1, wherein when the bladder member is moved from the collapsed position to the expanded position, the porous filter material is configured and dimensioned to substantially cover three major cerebral arteries in an aortic arch of the desired anatomical location.

5. The assembly of claim 1, wherein the porous filter material includes a plurality of pores, each pore of the plurality of pores having a pore size of from about 100 μm to about 150 μm.

6. The assembly of claim 1, wherein the bladder member is hollow and substantially tubular.

7. The assembly of claim 1, wherein a top side of the bladder member defines the window portion, the window portion rectangular in shape.

8. The assembly of claim 1 further comprising a top tether member and a bottom tether member attached to the distal end of the bladder member, the top tether member including a fill hose through which fluid is moved in order to inflate or deflate the bladder member; and
wherein the bottom tether member is attached to a support member, the support member extending from the distal end of the bladder member to the proximal end of the bladder member.

9. The assembly of claim 8, wherein the support member is a semi-rigid rod or wire that is attached to the bladder member at the distal end and the proximal end of the bladder member.

10. The assembly of claim 1, wherein the inner and outer walls of the bladder member include a plurality of sealed perforations therethrough.

11. A method for performing a procedure, comprising:
providing a bladder member extending from a proximal end to a distal end, the bladder member having an outer wall and an inner wall sealed together to form an inflatable cavity between the outer and inner walls, the bladder member defining a window portion, with a porous filter material mounted to the bladder member, the porous filter material extending across and covering the window portion;
positioning the bladder member in a desired anatomical location; and
introducing fluid to the bladder member to cause the bladder member to expand from a collapsed position to an expanded position.

12. The method of claim 11, wherein after the bladder member is positioned in the expanded position, removal of fluid from the bladder member causes the bladder member to collapse from the expanded position to the collapsed position.

13. The method of claim 11, wherein when the bladder member is expanded from the collapsed position to the expanded position, the outer wall of the expanded bladder member is configured and dimensioned to substantially conform to an aortic wall of the desired anatomical location.

14. The method of claim 11, wherein when the bladder member is moved from the collapsed position to the expanded position, the porous filter material is configured and dimensioned to substantially cover three major cerebral arteries in an aortic arch of the desired anatomical location.

15. The method of claim 11, wherein the porous filter material includes a plurality of pores, each pore of the plurality of pores having a pore size of from about 100 μm to about 150 μm.

16. The method of claim 11, wherein the bladder member is hollow and substantially tubular.

17. The method of claim 11, wherein a top side of the bladder member defines the window portion, the window portion rectangular in shape.

18. The method of claim 11 further comprising a top tether member and a bottom tether member attached to the distal end of the bladder member, the top tether member including a fill hose through which fluid is moved in order to inflate or deflate the bladder member; and
wherein the bottom tether member is attached to a support member, the support member extending from the distal end of the bladder member to the proximal end of the bladder member.

19. The method of claim 18, wherein the support member is a semi-rigid rod or wire that is attached to the bladder member at the distal end and the proximal end of the bladder member.

20. The method of claim 11, wherein the inner and outer walls of the bladder member include a plurality of sealed perforations therethrough.

21. A protection assembly comprising:
a hollow and tubular bladder member extending from a proximal end to a distal end, the bladder member having an outer wall and an inner wall sealed together to form an inflatable cavity between the outer and inner walls, a top side of the bladder member defining a rectangular window portion;
a porous filter material mounted to the bladder member, the porous filter material extending across and covering the rectangular window portion, the porous filter material including a plurality of pores, each pore of the plurality of pores having a pore size of from about 100 μm to about 150 μm;
a top tether member and a bottom tether member attached to the distal end of the bladder member, the top tether member including a fill hose through which fluid is moved in order to inflate or deflate the bladder member, and the bottom tether member attached to a support member, the support member extending from the distal end of the bladder member to the proximal end of the bladder member;
wherein after the bladder member is positioned in a desired anatomical location, introduction of fluid to the bladder member causes the bladder member to expand from a collapsed position to an expanded position;
wherein after the bladder member is positioned in the expanded position, removal of fluid from the bladder member causes the bladder member to collapse from the expanded position to the collapsed position;
wherein when the bladder member is expanded from the collapsed position to the expanded position, the outer wall of the expanded bladder member is configured and dimensioned to substantially conform to an aortic wall of the desired anatomical location; and
wherein when the bladder member is moved from the collapsed position to the expanded position, the porous filter material is configured and dimensioned to substantially cover three major cerebral arteries in an aortic arch of the desired anatomical location.

22. A protection assembly comprising:
a proximal cylindrical bladder member having an outer wall and an inner wall sealed together to form an inflatable cavity between the outer and inner walls;
a distal cylindrical bladder member having an outer wall and an inner wall sealed together to form an inflatable cavity between the outer and inner walls;
a cylindrical member mounted with respect to the proximal and distal bladder members, the cylindrical member extending from the proximal bladder member to the distal bladder member;
wherein after the proximal and distal bladder members are positioned in a desired anatomical location, introduction of fluid to the proximal and distal bladder members causes the proximal and distal bladder members to expand from a collapsed position to an expanded position.

23. (canceled)

24. (canceled)

25. (canceled)

26. (canceled)

27. (canceled)

28. (canceled)

29. The assembly of claim 22 further comprising first and second proximal filter members mounted to the proximal cylindrical bladder member and to the cylindrical member, and a distal filter member mounted to the distal cylindrical bladder member and to the cylindrical member.

30. The assembly of claim 29, wherein the first proximal filter member is attached along its outer and larger circumference to the proximal cylindrical bladder member, and is attached along its inner and smaller circumference to the extreme proximal end of the cylindrical member;
wherein the second proximal filter member is attached along its outer and larger circumference to the proximal cylindrical bladder member, and is attached along its inner and smaller circumference to a position along the proximal end of the cylindrical member; and
wherein the distal filter member is attached along its outer and larger circumference to the distal cylindrical bladder member, and is attached along its inner and smaller circumference to the extreme distal end of the cylindrical member.
